(19) United States
(12) Patent Application Publication
(10) Pub. No.: US 2004/0204463 A1
(43) Pub. Date: Oct. 14, 2004

(54) N-aryl-2-oxazolidinone-5-carboxamides and their derivatives

(76) Inventor: Christina Renee Harris, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/795,192

(22) Filed: Mar. 5, 2004

Related U.S. Application Data
(60) Provisional application No. 60/459,444, filed on Apr. 1, 2003.

(51) Int. Cl. C07D 413/02; C 07D 4 5/02; A61K 31/55; A61K 31/423

(52) U.S. Cl. 514/375; 514/380; 514/397; 548/229; 548/243; 548/315.4

(57) ABSTRACT

The present invention provides antibacterial agents having the formula I described herein.

### CROSS REFERENCE TO RELATED APPLICATIONS

This application claims the benefit of the following provisional application: Application Serial No. 60/459,444 filed Apr. 1, 2003 under 35 U.S.C. 119(e)(1).

The present invention relates to novel N-Aryl-2-oxazolidinone-5-carboxamides, derivatives thereof, and their preparations. These compounds have potent antibacterial activity.

### BACKGROUND OF THE INVENTION

The oxazolidinone antibacterial agents are a novel synthetic class of antimicrobials with potent activity against a number of human and veterinary pathogens, including Gram-positive aerobic bacteria such as multiply-resistant staphylococci and streptococci, anaerobic organisms such as bacteroides and clostridia species, and acid-fast organisms such as Mycobacterium tuberculosis and Mycobacterium avium.

### SUMMARY OF THE INVENTION

This invention provides compounds of Formula I.

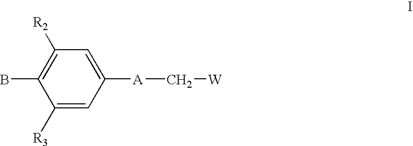

or pharmaceutically acceptable salts thereof wherein:

A is a structure i, ii, iii, or iv;

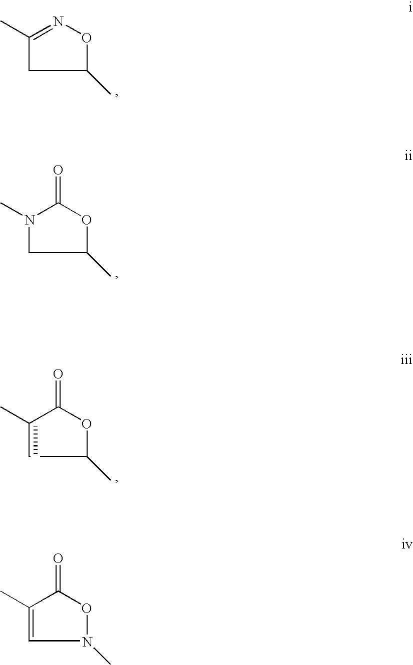

B is

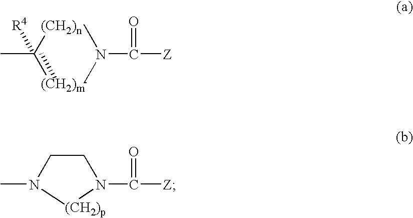

W is —N(H)C(═X)—R₁, Het, or —Y-HET, in which the Het or —Y-HET is optionally substituted with ═S or ═O;

X is O or S;

Y is NH, O, or S;

Z is

R₅—≡—(CH₂)ᵣ—E—

E is CH₂ or C═O;

R₁ is a) H,

b) NH₂,

c) NHC₁₋₄alkyl,

d) C₁₋₄ alkyl,

e) C₂₋₄ alkenyl,

f) O—C₁₋₄alkyl,

g) S—C₁₋₄ alkyl, or

h) (CH₂)ₛ C₃₋₆ cycloalkyl, in which each occurrence of alkyl or cycloalkyl in R₁ is optionally substituted by 1-3 halo;

Each R₂ and R₃ is independently hydrogen, halogen (F or Cl), methyl or ethyl;

R₄ is H, CH₃ or F;

R₅ is selected from H, aryl, and heteroaryl, each optionally substituted with 1-3 of R₆;

R₆ is halogen, (CH₂)ₘNHR₇, (CH₂)ₚR₇, CH₂—CHR₉—C(O)—R₈, OR₈, S(O)_{q}R₇, CN, C(═O)R₉, C(═NR₁₀)NHR₈, or C(═NR₁₀)R₈;

Each R₇ and R₈ is independently H, C₁₋₆ alkyl, aryl, or heteroaryl;

R₉ is OH, OR₈, C₁₋₆ alkyl, aryl, heteroaryl, or N(R₇)(R₈);

R₁₀ is OR₈ or N(R₇)(R₈);

m is 0, 1, 2, 3, 4;

n is 0, 1, 2, 3, 4 with the proviso that m plus n is 2, 3, 4, or 5;

p is 1, 2, 3;

q is 0, 1, 2;

r and s are independently 0, 1, 2, 3, 4, 5 or 6.

Embodiments of the invention may include one or more of the following. r is 1, 2, 3, or 4. R₅ is phenyl optionally substituted with R₆. R₆ is (CH₂)ₙNHR₇, such as —CH₂—NH₂. R₆ is CH₂—CHR₉—C(O)—R₈, such as —CH₂—CH(NH₂)—C(O)—OH or —CH₂—CH(NH₂)—C(O)—O—CH₃. R₆ is OR₈, such as —OH or —OCH₃. R₆ is C(═O)R₉, such as —C(O)—CH₃.

Specific compounds of the invention include:

N-({(5S)-3-[4-(4-{6-[3-(aminomethyl)phenyl]hex-5-ynoyl}piperazin-1-yl)-3-fluorophenyl]-2-oxo-1,3-oxazolidin-5-yl}methyl)acetamide;

N-({(5S)-3-[3-fluoro-4-(4-hex-5-ynoylpiperazin-1-yl)phenyl]-2-oxo-1,3-oxazolidin-5-yl)}methyl)acetamide;

N-({(5S)-3-[3-fluoro-4-(4-hept-6-ynoylpiperazin-1-yl)phenyl]-2-oxo-1,3-oxazolidin-5-yl}methyl)acetamide;

N-{[(5S)-3-(3-fluoro-4-{4-[5-(4-hydroxyphenyl)pent-4-ynoyl]piperazin-1-yl}phenyl)-2-oxo-1,3-oxazolidin-5-yl]methyl}acetamide;

N-{[(5S)-3-(3-fluoro-4-{4-[5-(3-hydroxyphenyl)pent-4-ynoyl]piperazin-1-yl}phenyl)-2-oxo-1,3-oxazolidin-5-yl]methyl}acetamide;

N-({(5S)-3-[4-(4-{5-[4-(aminomethyl)phenyl]pent-4-ynoyl}piperazin-1-yl)-3-fluorophenyl]-2-oxo-1,3-oxazolidin-5-yl}methyl)acetamide;

N-{[(5S)-3-(3-fluoro-4-{4-[6-(4-hydroxyphenyl)hex-5-ynoyl]piperazin-1-yl}phenyl)-2-oxo-1,3-oxazolidin-5-yl]methyl}acetamide;

N-{[(5S)-3-(3-fluoro-4-{4-[6-(3-hydroxyphenyl)hex-5-ynoyl]piperazin-1-yl}phenyl)-2-oxo-1,3-oxazolidin-5-yl]methyl}acetamide;

N-({(5S)-3-[4-(4-{6-[4-(aminomethyl)phenyl]hex-5-ynoyl}piperazin-1-yl)-3-fluorophenyl]-2-oxo-1,3-oxazolidin-5-yl}methyl)acetamide;

N-{[(5S)-3-(3-fluoro-4-{4-[7-(3-hydroxyphenyl)hept-6-ynoyl]piperazin-1-yl}phenyl)-2-oxo-1,3-oxazolidin-5-yl]methyl}acetamide;

N-{[(5S)-3-(3-fluoro-4-{4-[7-(4-hydroxyphenyl)hept-6-ynoyl]piperazin-1-yl}phenyl)-2-oxo-1,3-oxazolidin-5-yl]methyl}acetamide;

N-({(5S)-3-[4-(4-{7-[4-(aminomethyl)phenyl]hept-6-ynoyl}piperazin-1-yl)-3-fluorophenyl]-2-oxo-1,3-oxazolidin-5-yl}methyl)acetamide;

N-{[(5S)-3-(4-{4-[5-(2,4-dioxo-1,2,3,4-tetrahydropyrimidin-5-yl)pent-4-ynoyl]piperazin-1-yl}-3-fluorophenyl)-2-oxo-1,3-oxazolidin-5-yl]methyl}acetamide;

Methyl-4-{5-[4-(4-{(5S)-5-[(acetylamino)methyl]-2-oxo-1,3-oxazolidin-3-yl}-2-fluorophenyl)piperazin-1-yl]-5-oxopent-1-ynyl}-L-phenylalaninate;

N-{[(5S)-3-(4-{4-[5-(4-aminophenyl)pent-4-ynoyl]piperazin-1-yl}-3-fluorophenyl)-2-oxo-1,3-oxazolidin-5-yl]methyl}acetamide;

N-[((5S)-3-{3-fluoro-4-[4-(6-{4-[(methylamino)methyl]phenyl} hex-5-ynoyl)piperazin-1-yl]phenyl}-2-oxo-1,3-oxazolidin-5-yl)methyl]acetamide;

N-({(5S)-3-[3-fluoro-4-(4-{6-[4-(1H-imidazol-1-ylmethyl)phenyl]hex-5-ynoyl}piperazin-1-yl)phenyl]-2-oxo-1,3-oxazolidin-5-yl}methyl)acetamide; N-{[(5S)-3-(4-{4-[6-(4-acetylphenyl)hex-5-ynoyl]piperazin-1-yl}-3-fluorophenyl)-2-oxo-1,3-oxazolidin-5-yl]methyl}acetamide;

N-({(5S)-3-[4-(1-{6-[3-(aminomethyl)phenyl]hex-5-ynoyl}-3-methylazetidin-3-yl)phenyl]-2-oxo-1,3-oxazolidin-5-yl}methyl)propanamide;

N-[((5S)-3-{3-fluoro-4-[4-(6-{4-[(1 E)-N-hydroxyethanimidoyl]phenyl} hex-5-ynoyl)piperazin-1-yl]phenyl}-2-oxo-1,3-oxazolidin-5-yl)methyl]acetamide;

N-{[(5S)-3-(4-{4-[6-(3-cyanophenyl)hex-5-ynoyl]piperazin-1-yl }-3-fluorophenyl)-2-oxo-1,3-oxazolidin-5-yl]methyl}acetamide;

4-{5-[4-(4-{(5S)-5-[(acetylamino)methyl]-2-oxo-1,3-oxazolidin-3-yl}-2-fluorophenyl)piperazin-1-yl]-5-oxopent-1-ynyl}-L-phenylalanine;

N-[((5S)-3-{4-[4-(6-{4-[(Z)-amino(hydroxyimino)methyl]phenyl}hex-5-ynoyl)piperazin-1-yl]-3-fluorophenyl}-2-oxo-1,3-oxazolidin-5-yl)methyl]acetamide hydrochloride;

N-({(5S)-3-[4-(4-{6-[3-(aminomethyl)phenyl]hex-5-ynoyl}piperazin-1-yl)-2,3,5-trifluorophenyl]-2-oxo-1,3-oxazolidin-5-yl}methyl)acetamide and

N-({(5S)-3-[4-(4-{6-[4-(aminomethyl)phenyl]hex-5-ynoyl}piperazin-1-yl)-2,3,5-trifluorophenyl]-2-oxo-1,3-oxazolidin-5-yl}methyl)acetamide.

Compounds of Formula I have antibacterial activity against a number of human and veterinary pathogens including Gram-positive aerobic bacteria such as multiply-resistant-staphylococci, streptococci and enterococci, Gram-negative organisms such as H. influenzae and M. catarrhalis, anaerobic organisms such as Bacteroides spp. and clostridia spp., Mycobacterium tuberculosis, M. avium and M. spp. and in organisms such as Mycoplasma spp. For use as antibacterial agents the compounds of this invention can be administered orally or parenterally in a dosage range of about 0.1-100 mg/kg or preferably of about 1.0-50 mg/kg of body weight per day. Advantageously, the compound of the invention exhibit antibacterial activity against S. aureus resistant organisms.

### DETAILED DESCRIPTION OF THE INVENTION

The following definitions are used, unless otherwise described.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term “halo” refers to a halogen atom selected from Cl, Br, I, and F.

The term “alkyl” refers to both straight- and branched-chain moieties. Unless otherwise specifically stated alkyl moieties include between 1 and 6 carbon atoms.

The term “alkenyl” refers to both straight- and branched-chain moieties containing at least one —C═C—. Unless otherwise specifically stated alkenyl moieties include between 1 and 6 carbon atoms.

The term “alkoxy” refers to —O-alkyl groups.

The term “cycloalkyl” refers to a cyclic alkyl moiety. Unless otherwise specifically stated cycloalkyl moieties will include between 3 and 7 carbon atoms.

The term “amino” refers to —NH₂.

The term “aryl” refers to phenyl and naphthyl.

The term “het” refers to mono- or bicyclic ring systems containing at least one heteroatom selected from O, S, and N. Each monocyclic ring may be aromatic, saturated, or partially unsaturated. A bicyclic ring system may include a monocyclic ring containing at least one heteroatom fused with a cycloalkyl or aryl group. A bicyclic ring system may also include a monocyclic ring containing at least one heteroatom fused with another het, monocyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heterocycle” refers to a fully saturated het, examples of which include, but are not limited to, morpholinyl, thiomorpholinyl, and tertrahydropyranyl.

Specific R₃ and R₄ substituents include H, F, Cl, Br, CN, NH₂, NO₂, CH₃.

Specific structures of A include

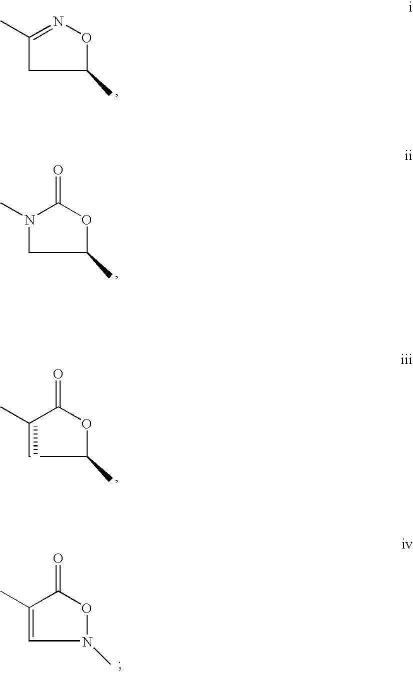

Mammal refers to human or animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “O” for oxygen atom, “S” for sulfur atom, “N” for nitrogen atom, “h” for hour or hours and “rt” for room temperature) as described in J.Org.Chem., 67-1, 24A, 2002.

Other abbreviations and definitions used are defined as follows:

Hunig's base means diisopropylethyl amine;

HATU means O-(7-Azabenzotriazol-1-yl)-N,N,N′,N′-tetramethyluronium hexaflourophosph ate; in vacuo means at reduced pressure;

EDCI or EDC means 1-ethyl-3-(3-dimethylaminopropyl)carbodimide;

HOBT means hydroxybenztriazole;

Fmoc means 9-fluorenylmethoxycarbonyl;

trisamine resin means tris(2-aminoethyl)amine, polymer-bound;

DPPA means diphenylphosphoryl azide.

The compounds of the present invention can be converted to their salts, where appropriate, according to conventional methods.

The term “pharmaceutically acceptable salts” refers to acid addition salts useful for administering the compounds of this invention and include hydrochloride, hydrobromide, hydroiodide, sulfate, phosphate, acetate, propionate, lactate, mesylate, maleate, malate, succinate, tartrate, citric acid, 2-hydroxyethyl sulfonate, fumarate and the like. These salts may be in hydrated form.

The compounds of Formula I of this invention contain a chiral center, such as at C-5 of the isoxazoline ring, and as such there exist two enantiomers or a racemic mixture of both. This invention relates to both the enantiomers, as well as mixtures containing both the isomers. In addition, depending on substituents, additional chiral centers and other isomeric forms may be present in any of A or R₁ group, and this invention embraces all possible stereoisomers and geometric forms in these groups.

The compounds of this invention are useful for treatment of microbial infections in humans and other warm blooded animals, under both parenteral and oral administration.

The pharmaceutical compositions of this invention may be prepared by combining the compounds of this invention with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds of this invention dissolved in water and water-propylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component, that is, the compound according to this invention.

The quantity of active component, that is the compound according to this invention, in the pharmaceutical composition and unit dosage form thereof may be varied or adjusted widely depending upon the particular application, the potency of the particular compound and the desired concentration. Generally, the quantity of active component will range between 0.5% to 90% by weight of the composition.

In therapeutic use for treating, or combatting, bacterial infections in warm-blooded animals, the compounds or pharmaceutical compositions thereof will be administered orally, parenterally and/or topically at a dosage to obtain and maintain a concentration, that is, an amount, or blood-level of active component in the animal undergoing treatment which will be antibacterially effective. Generally, such antibac-terially effective amount of dosage of active component will be in the range of about 0.1 to about 100, more preferably about 1.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2-4 four times per day.

The compounds according to this invention may be administered parenterally, i.e., by injection, for example, by intravenous injection or by other parenteral routes of administration. Pharmaceutical compositions for parenteral administration will generally contain a pharmaceutically acceptable amount of the compound or a soluble salt (acid addition salt or base salt) dissolved in a pharmaceutically acceptable liquid carrier such as, for example, water-for-injection and a buffer to provide a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine to name but a few representative buffering agents. The compounds of this invention generally will be dissolved in the carrier in an amount sufficient to provide a pharmaceutically acceptable injectable concentration in the range of about 1 mg/mL to about 400 mg/mL of solution. The resulting liquid pharmaceutical composition will be administered so as to obtain the above-mentioned antibacterially effective amount of dosage. The compounds according to this invention are advantageously administered orally in solid and liquid dosage forms.

As a topical treatment an effective amount of Formula I is admixed in a pharmaceutically acceptable gel or cream vehicle that can be applied to the patient's skin at the area of treatment. Preparation of such creams and gels is well known in the art and can include penetration enhancers.

The oxazolidinone antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

Compounds in this invention can be prepared as shown in Scheme I. In Scheme I an amine (1) is condensed with a suitably substituted alkyne carboxylic acid (HOOC-Z′) to give an amide (2) where Z′ represents Z of formula I in which R₅ is H. Amide 2 is coupled with R₅-L, where L represents a suitable coupling group such as halogen, trifluromethanesulfonate or the like. R₅-L may contain a suitable protecting group for hydroxy or amino substitutents that can be removed at an appropriate time in a manner that is compatible with other substituents on the molecule to give the title compound (3). A variety of reagents and reaction condensations can be used for the condensations of 1 with the carboxylic acids (HOO-Z′). These include but are not limited to the carbodiimides such as dicyclohexylcarbodiimide (DCC) and 1-(3-dimethylaminopropyl)-3-ethylcarbodiimide hydrochloride (EDC) which can be used with promoters such as 4-(dimethylamino)pyridine (DMAP) or I-hydroxybenzotriazole (HOBT) in solvents such as THF, DMF or pyridine at 0° C. to 50° C. The terminal alkynes 2 can be coupled via a protocol using either PdCl₂(PPh₃)₂ or Pd(dppf)Cl₂ in the presence of Ph₃As, CuI, and Et₃N in DMF solvent at room temperature as has been described (de Kort, M., et.al., J. Med. Chem. 2000, 43, 3295).

By using the chemistry described in Scheme I and employing other side chains known in the literature or described in the examples other compounds of formula I can be prepared.

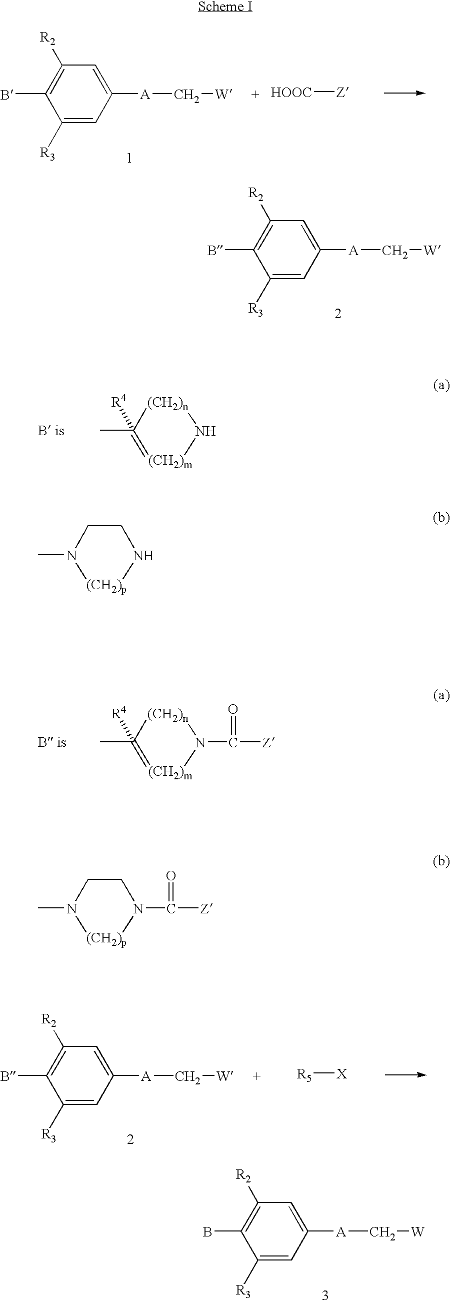

Suitable intermediates useful in preparing compounds of formula I and additional synthetic methods to assist in producing compounds of formula I may be found, for example, in the following publications each of which is hereby incorporated by reference.

U.S. Pat. Nos. 5,225,565; 5,182,403; 5,164,510; 5,247,090; 5,231,188; 5,565,571; 5,547,950; 5,529,998; 5,627,181; 5,843,967; 5,861,413; 5,827,857; 5,869,659; 5,952,324; 5,968,962; 5,688,792; 6,069,160; 6,239,152; 5,792,765; 4,705,799; 5,043,443; 5,652,238; 5,827,857; 5,529,998; 5,684,023; 5,627,181; 5,698,574; 6,166,056; 6,194,441; 6,110,936; 6,069,145; 6,271,383; 5,981,528; 6,051,716; 6,043,266; 6,313,307; and 5,523,403.

U.S. Patent Application Publication 2002/0086900.

PCT Application and publications PCT/US93/04850, WO94/01110; PCT/US94/08904, WO95/07271; PCT/US95/02972, WO95/25106; PCT/US95/10992, WO96/13502; PCT/US96/05202, WO96/35691; PCT/US96/12766; PCT/US96/13726; PCT/US96/14135; PCT/US96/17120; PCT/US96/19149; PCT/US97/01970; PCT/US95/12751, WO96/15130, PCT/US96/00718, WO96/23788, WO98/54161, WO99/29688, WO99/03846, WO99/37641, WO99/37652, WO99/40094, WO97/30995, WO97/09328, WO01/81350, WO01/40236, WO00/21960 WO01/4022, and WO95/07271.

In some embodiments, the antibacterial compounds are prodrugs of the compounds of formula I. The expression “prodrug” denotes a derivative of a known direct acting drug, which is transformed into the active drug by an enzymatic or chemical process. Prodrugs of the compounds of formula I are prepared by modifying functional groups present on the compound in such a way that the modifications are cleaved, either in routine manipulation or in vivo, to the parent compound. Prodrugs include, but are not limited to, compounds of structure (I) wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### N-({(5S)-3-[3-Fluoro-4-(4-hex-5-ynoylpiperazin-1-yl)phenyl]-2-oxo-1,3-oxazolidin-5-yl}methyl)acetamide.

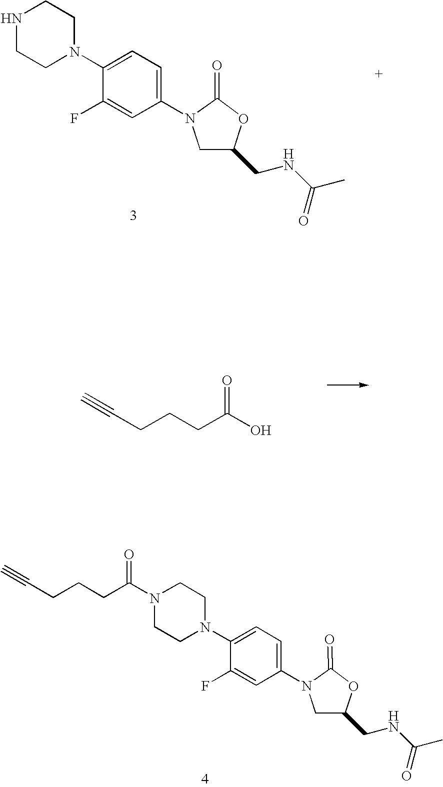

Amine 3 (U.S. Patent Application Publication 2002/86900) (3.0 g, 5.36 mmol), 5-hexynoic acid (0.66 g, 5.90 mmol), and Hunig's base (5 mL) were cooled to 0° C. in an ice bath and then diphenylphosphoryl azide (1.62 g, 5.90 mmol) was added dropwise via syringe. The reaction mixture was allowed to warm slowly to rt and then stirred 14 h. The reaction mixture was diluted with EtOAc and water added. The water layer was extracted five times with EtOAc, the organic layers were combined and washed with water, saturated aqueous NaHCO₃, brine and dried over MgSO₄. The extract was concentrated in vacuo and then triturated with Et₂O to afford the desired amide 4; ¹H NMR (300 MHz, DMSO-d6) δ 8.24 (m, 1H), 7.50 (dd, J=16, 2 Hz, 1H), 7.18 (dd, J=2, 8 Hz, 1H), 7.07 (dd, J=12, 12 Hz, 1H), 4.71 (m, 1H), 4.08 (m, 1H), 3.70 (dd, J=8, 6 Hz, 1H), 3.60 (m, 4H), 3.40 (m, 2H), 2.98-2.89 (m, 4H), 2.81 (m, 1H), 2.44 (t, J=4 Hz, 2H), 2.21 (dt, J=4, 8 Hz, 2H), 1.83 (s, 3H), 1.69 (m, 2H).

### Example 2

### N-({(5S)-3-[4-(4-{6-[3-(aminomethyl)phenyl]hex-5-ynoyl}piperazin-1-yl)-3-fluorophen-yl]-2-oxo-1,3-oxazolidin-5-yl}methyl)acetamide, 6.

Step 1: tert-Butyl 3-{6-[4-(4-{(5S)-5-[(acetylamino)methyl]-2-oxo-1,3-oxazolidin-3-yl}-2-fluorophenyl)piperazin-1-yl]-6-oxohex-1-ynyl}phenylcarbamate, 5.

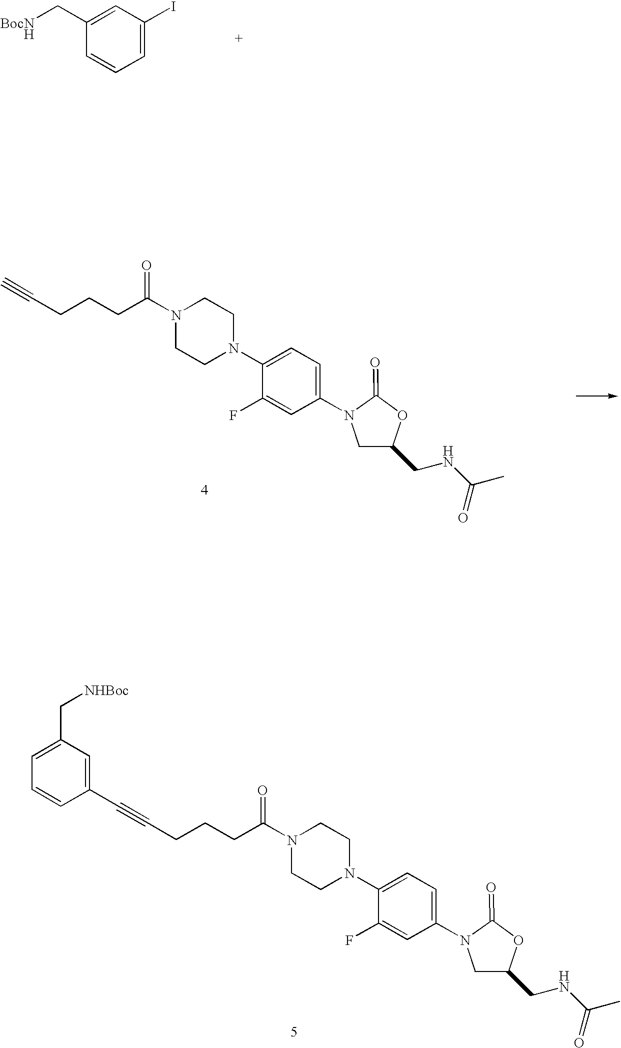

tert-Butyl 3-iodobenzylcarbamate (0.67 mmol) was dissolved in 1 mL of dry DMF and the resultant solution was evacuated to 40 mm pressure and then released to N₂ (g) three times. Triethylamine (1:8 ratio with DMF, 100 μL), Pd(PPh₃)₂Cl₂ (0.030 mmol), and CuI (0.067 mmol) were added. The resultant mixture evacuated to 40 mm pressure for Ca. 30 seconds and then released to N₂ (g). Finally, the alkyne 4 (0.67 mmol) in DMF (0.07 M) was added dropwise via an addition funnel over 40 minutes. The reaction mixture was stirred at room temperature for 18 hr and then diluted with ethyl acetate, poured into 0.1 M HCl and extracted with EtOAc (3×100 mL). The combined organic layers were washed with water and brine and dried over MgSO₄. The extract was concentrated and the product purified by Biotage chromatography (40M, product loaded onto SiO₂) with 2% MeOH/CH₂Cl₂ to afford the desired title compound: ¹H NMR (400 MHz, DMSO-d₆) δ 8.24 (m, 1H), 7.50 (dd, J=4, 16 Hz, 1 H), 7.41 (m, 1H), 7.26 (m, 3H), 7.18 (m, 2H), 7.05 (m, 1H), 4.71 (m, 1H), 4.08 (m, 3H), 3.70 (dd, J=4, 12 Hz, 1H), 3.62 (m, 4H), 3.40 (m, 2H), 2.92 (m, 4H), 2.47 (m, 4H), 1.83 (s, 3H), 1.79 (m, 2H), 1.38 (s, 9H).

Step 2: N-({(5S)-3-[4-(4-{6-[3-(aminomethyl)phenyl]hex-5-ynoyl}piperazin-1-yl)-3-fluorophen-yl]-2-oxo-1,3-oxazolidin-5-yl}methyl)acetamide, 6.

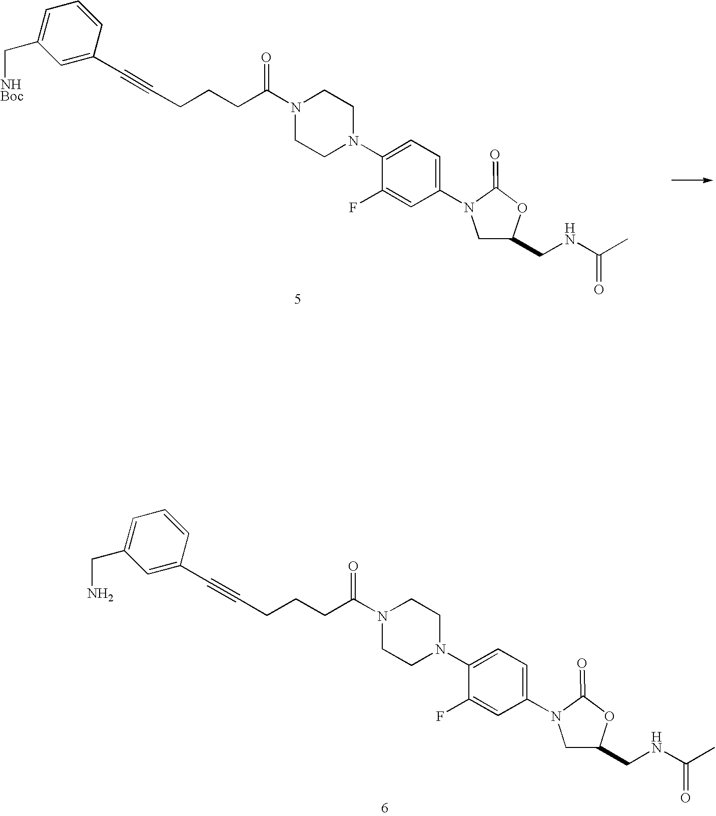

The NBoc amine (5) was suspended in CH₂Cl₂ (ca. 5 mL) and treated with TFA (4 mL) at 0° C. to rt for 1.5 hr. The reaction mixture was concentrated in vacuo and subjected to Biotage chromatography with 5% MeOH/CH₂Cl₂/NH₃ to afford the free amine 6: ¹H NMR (300 MHz, DMSO-d₆) δ 8.24 (m, 1H), 7.50 (dd, J=15, 3 Hz, 1H), 7.39 (m, 1H), 7.29-7.22 (m, 3H), 7.17 (dd, J=9, 6 Hz, 1H), 7.05 (m, 1H), 4.71 (m, 1H), 4.08 (t, J=9 Hz, 1H), 3.71 (m, 3H), 3.62 (m, 4H), 3.40 (m, 2H), 3.25 (bs, 2H), 2.93 (m, 4H), 2.47 (m, 2H), 1.83 (s, 3H), 1.79 (m, 2H).

### Example 3

### N-{[(5S)-3-(3-Fluoro-4-{4-[6-(4-hydroxyphenyl)hex-5-ynoyl]piperazin-1-yl}phenyl)-2-oxo-1,3-oxazolidin-5-yl]methyl}acetamide, 7.

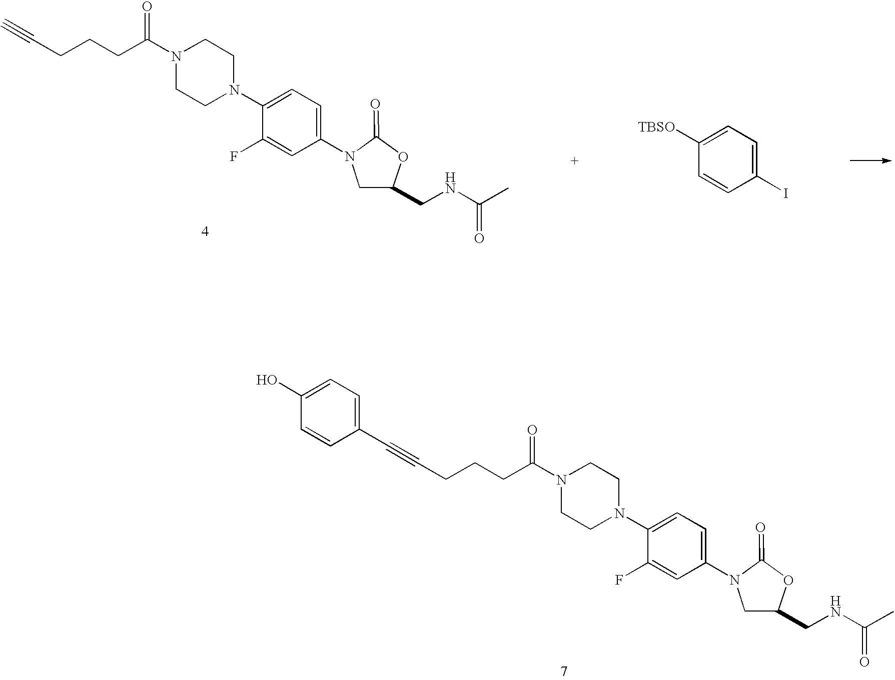

The title compound was prepared by coupling 4 (1.20 g, 2.79 mmol) with 4-t-butyldimethylsilyloxy-p-iodophenol (1.12 g, 3.34 mmol) as described in example 2, step 1, and deprotection of the intermediate silyl phenol with TBAF (3.0 mL of a 1.0 M solution in THF) in THF at 0° C. for 4 hr to afford the title compound 7. ¹H NMR (300 MHz, DMSO-d₆) δ 9.72 (s, 1H), 8.24 (m, 1H), 7.50 (dd, J=2, 16 Hz, 1H), 7.20 (d, J=12 Hz, 2H), 7.18 (m, 1H), 7.06 (dd, J=12, 12 Hz, 1H), 6.71 (d, J=8 Hz, 2H), 4.71 (m, 1H), 4.08 (t, J=8 Hz, 1H), 3.70 (dd, J=8, 12 Hz, 1H), 3.62 (m, 4H), 3.40 (t, J=4 Hz, 2H), 2.92 (m, 4H), 2.49 (dd, J=8, 8 Hz, 2H), 2.42 (dd, J=8, 8, 2H), 1.83 (s, 3H), 1.76 (m, 2H).

### Example 4

### N-[((5S)-3-{3-fluoro-4-[4-(4-pentynoyl)-1-piperazinyl]phenyl}-2-oxo-1,3-oxazolidin-5-yl)methyl]acetamide, 8.

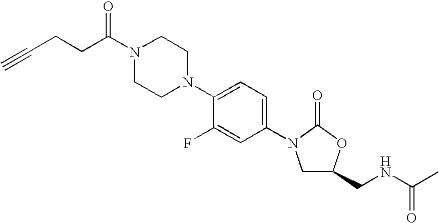

Prepared as in Example 1 with nonessential modifications but substituting pentynoic acid for hexynoic acid. ¹H NMR (400 MHz, DMSO-d₆) d 8.24 (t, J=4 Hz, 1H), 7.50 (dd, J=2, 16 Hz, 1H), 7.18 (dd, J=2, 8 Hz, 1H), 7.07 (t, J=8 Hz, 1H), 4.71 (m, 1H), 4.08 (t, J=8 Hz, 1H), 3.70 (dd, J=8, 12 Hz, 1H), 3.60 (m, 4H), 3.40 (t, J=4 Hz, 2H), 2.97 (m, 2H), 2.91 (m, 2H), 2.78 (t, J=4 Hz, 1H), 2.59 (t, J=4 Hz, 2H), 2.83 (dt, J=4, 8 Hz, 2H), 1.83 (s, 3H).

### Example 5

### N-[((5S)-3-{3-fluoro-4-[4-(3-phenylprop-2-ynyl)piperazin-1-yl]phenyl}-2-oxo-1,3-oxazolidin-5-yl)methyl]acetamide, 9.

Step 1: N-({(5S)-3-[3-fluoro-4-(4-prop-2-ynylpiperazin-1-yl)phenyl]-2-oxo-1,3-oxazolidin-5-yl} methyl)acetamide, 9A

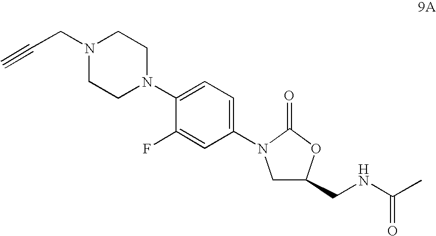

N-({(5S)-3-[3-fluoro-4-(1-piperaziny-1)phenyl]-2-oxo-1,3-oxazolidin-5-yl}methyl)acetamide hydrochloride (8.0 g, 21.4 mmol) was dissolved in DMF and treated with K₂CO₃ (15.0 g, 107 mmol) for 1 hr. After this period of time, the reaction was cooled to 0° C. and propargyl bromide (4.78 g of an 80% weight solution in toluene, 32.4 mmol) was added. The cooling bath was allowed to expire and the reaction was stirred 18 hr at rt at which time, TLC showed complete consumption of the starting material and formation of a new, single, higher Rf product. The reaction was quenched with water and the organic product extracted with EtOAc. The combined organic layers were washed with brine, dried over MgSO₄, and concentrated in vacuo to afford a yellow oil, which was purified by chromatography with 3% MeOH:CH₂Cl₂. ¹H NMR (400 MHz, DMSO-d₆) δ 8.24 (t, J=4 Hz, 1H), 7.48 (dd, J=1, 12 Hz, 1H), 7.16 (m, 1H), 7.06 (t, J=8 Hz, 1H), 4.70 (m, 1H), 4.08 (t, J=8 Hz, 1H), 3.70 (dd, J=4, 6 Hz, 1H), 3.40 (m, 2H), 3.19 (m, 1H), 2.99 (m, 4H), 2.61 (m, 4H), 1.83 (s, 3H).

Step 2: N-[((5S)-3-{3-Fluoro-4-[4-(3-phenylprop-2-ynyl)piperazin-1-yl]phenyl}-2-oxo-1,3-oxazolidin-5-yl)methyl]acetamide, 9.

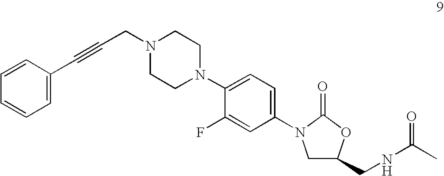

Prepared as in Example 2 with nonessential modifications but substituting the appropriate reactants; NMR (300 MHz, DMSO-d₆) δ 8.24 (m, 1H), 7.51-7.37 (m, 6H), 7.17 (dd, J=9, 3 Hz, 1 H), 7.07 (m, 1H), 4;71 (m, 1H), 4.08 (t, J=9 Hz, 1H), 3.70 (dd, J=6, 9 Hz, 1H), 3.59 (m, 2H), 3.40 (m, 2H), 3.03 (m, 4H), 2.70 (m, 4H), 1.83 (s, 3H).

### Example 6

### N-({(5S)-3-[4-(4-{6-[4-(aminomethyl)phenyl]hex-5-ynoyl}piperazin-1-yl)-2,3,5-trifluorophenyl]-2-oxo-1,3-oxazolidin-5-yl}methyl)acetamide, 10.

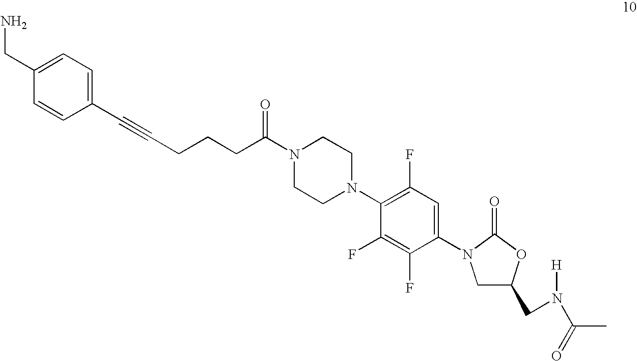

Prepared as in Example 2 with nonessential modifications but substituting the appropriate reactants. 1H NMR (300 MHz, DMSO-d₆) δ 8.26 (t, J=6.03 Hz, 1H), 7.36 (m, 2H), 7.24 (m, 1H), 7.31 (d, J=3.39 Hz, 2H), 4.76 (m, 1H), 4.06 (t, J=8.67 Hz, 1H), 3.70 (s, 2H), 3.69 (m, 1H), 3.58 (m, 4H), 3.42 (m, 2H), 3.34 (bs, 2H), 3.13 (m, 4H), 2.48 (m, 4H), 1.85 (s, 3H), 1.79 (m, 2H).

### Example 7

### N-({(5S)-3-[4-(4-{6-[3-(aminomethyl)phenyl]hex-5-ynoyl}piperazin-1-yl)-2,3,5-trifluorophenyl]-2-oxo-1,3-oxazolidin-5-yl}methyl)acetamide, 11.

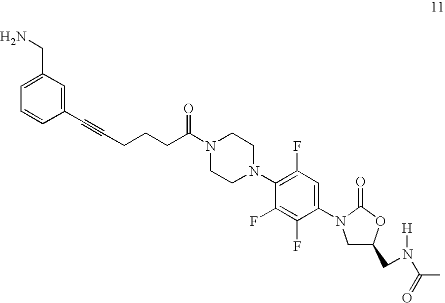

Prepared as in Example 2 with nonessential modifications but substituting the appropriate reactants. ¹H NMR (400 MHz, DMSO-d₆) δ 8.26 (t, J=5.84 Hz, 1H), 7.38 (m, 1H), 7.33 (m, 1H), 7.27 (m, 2H), 7.23 (m, 1H), 4.75 (m, 1H), 4.06 (t, J=8.67 Hz, 1H), 3.71 (m, 1H), 3.69 (s, 2H), 3.58 (m, 4H), 3.41 (m, 2H), 3.13 (m, 4H), 2.79 (bs, 2H), 2.45 (m, 4H), 1.85 (s, 3H), 1.79 (m, 2H);

### Example 8

### N-[((5S)-3-{4-[4-(6-{4-[(Z)-amino(hydroxyimino)methyl]-phenyl}hex-5-ynoyl)piperazin-1-yl]-3-fluorophenyl}-2-oxo-1,3-oxazolidin-5-yl)methyl]-acetamide hydrochloride, 12.

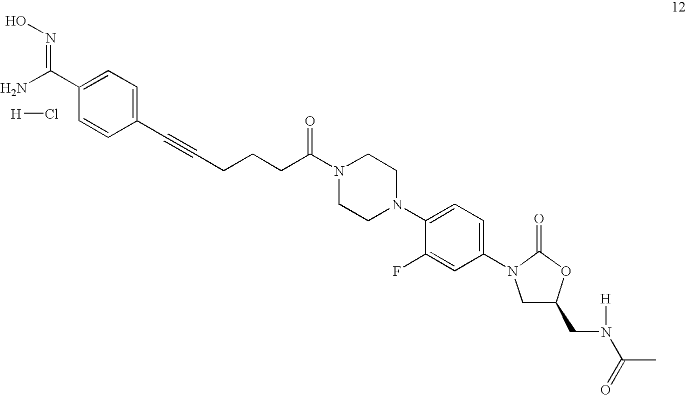

N-{[(5S)-3-(4-{4-[6-(3-cyanophenyl)hex-5-ynoyl]piperazin-1-yl}-3-fluorophenyl)-2-oxo-1,3-oxazolidin-5-yl]methyl}acetamide (0.300 g, 0.56 mmol) and hydroxylamine.HCl (86 mg, 1.24 mmol) in Et3N (1.29 mmol) and EtOH (2 mL) were heated at 75° C. The reaction was monitored by TLC and found to be complete after 12 hr. The reaction was diluted with water and taken to pH 7.0 with conc. HCl and the crude product was purified by chromatography (5-6% MeOH:CH₂Cl₂ to afford the HCl.salt. ¹H NMR (400 MHz, DMSO-D₆) □ ppm 1.80 (d, J=7.26 Hz, 2H), 1.83 (s, 3H), 2.93 (m, 4H), 3.17 (d, J=5.18 Hz, 1H), 3.33 (s, 4H), 3.40 (t, J=5.49 Hz, 2H), 3.62 (d, J=4.56 Hz, 4H), 3.70 (dd, J=9.23, 6.32 Hz, 1H), 4.08 (m, 1H), 4.70 (m, 1H), 5.87 (s, 2H), 7.05 (t, J=9.33 Hz, 1H), 7.17 (dd, J=8.81, 2.18 Hz, 1H), 7.37 (m, 2H), 7.50 (dd, J=14.72, 2.49 Hz, 1H), 7.64 (m, 1H), 7.69 (s, 1H), 8.24 (t, J=5.81 Hz, 1H), 9.70 (s, 1H).

### Example 9

### 4-{5-[4-(4-{(5S)-5-[(acetylamino)methyl]-2-oxo-1,3-oxazolidin-3-yl}-2-fluorophenyl) piperazin-1-yl]-5-oxopent-1-ynyl}-L-phenylalanine, 13.

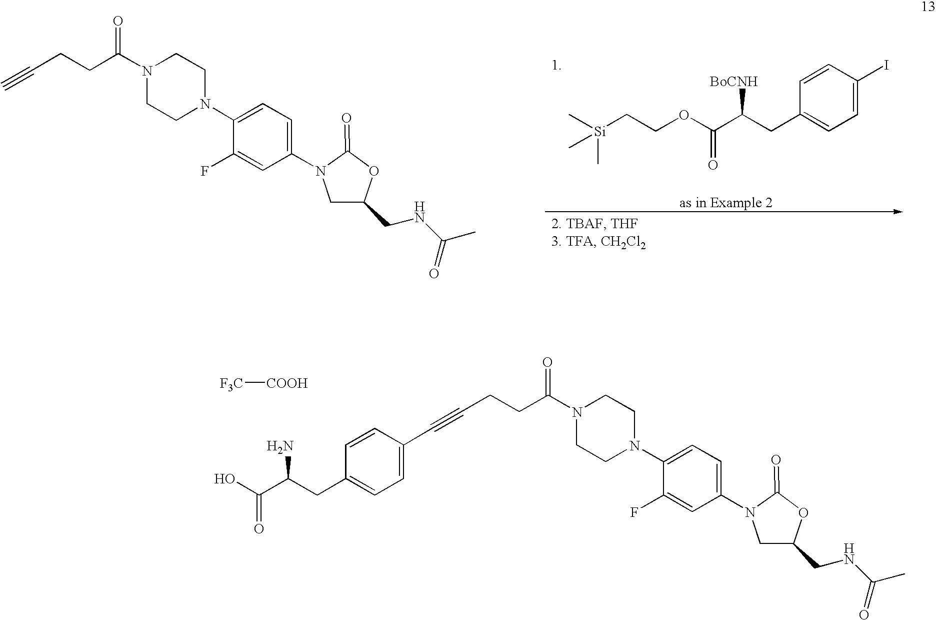

Prepared as in Example 2 with nonessential modifications but substituting the appropriate reactants. Subsequently, the silyl protecting group was removed with TBAF as in Example 3, and the Boc protecting group removed with TFA as described in Example 2. ¹H NMR (400 MHz, DMSO-d₆) □ ppm 1.84 (s, 3H), 2.66 (s, 4H), 2.84 (dd, J=13.99, 7.98 Hz, 1H), 2.94 (d, J=20.11 Hz, 4H), 3.12 (dd, J=13.89, 4.15 Hz, 1H), 3.33 (s, 5H), 3.39 (m, J=6.01 Hz, 2H), 3.63 (s, 4H), 3.73 (dd, J=8.81, 6.74 Hz, 1H), 4.09 (t, J=8.81 Hz, 1H), 4.70 (m, 1H), 7.01 (t, J=9.02 Hz, 1H), 7.15 (dd, J=8.60, 1.76 Hz, 1H), 7.22 (d, J=8.09 Hz, 2H), 7.28 (m, 2H), 7.52 (dd, J=14.82, 2.18 Hz, 1H), 8.34 (t, J=6.63 Hz, 1H).

### Example 10

### N-{[(5S)-3-(4-{4-[6-(3-cyanophenyl)hex-5-ynoyl]piperazin-1-yl}-3-fluorophenyl)-2-oxo-1,3-oxazolidin-5-yl]methyl}acetamide, 14.

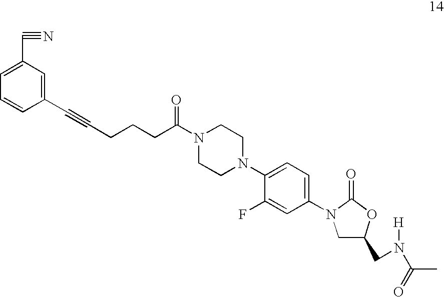

Prepared as in Example 2 with nonessential modifications but substituting the appropriate reactants (commerically available (3-cyano)iodobenzene was used in the Sonogashira coupling reaction). ¹H NMR (400 MHz, DMSO-d₆) □ ppm 1.79 (m, 2 H), 1.83 (s, 3H), 2.53 (d, J=3.52 Hz, 4H), 2.93 (m, 4H), 3.40 (t, J=5.49 Hz, 2H), 3.62 (m, 4H), 3.70 (dd, J=9.12, 6.43 Hz, 1H), 4.08 (m, 1H), 4.70 (m, 1H), 7.06 (t, J=9.33 Hz, 1H), 7.17 (m, 1H), 7.50 (dd, J=14.93, 2.49 Hz, 1H), 7.56 (t, J=7.88 Hz, 1H), 7.74 (dt, J=7.93, 1.43 Hz, 1H), 7.81 (dt, J=7.83, 1.37 Hz, 1H), 7.90 (t, J=1.45 Hz, 1H), 8.24 (t, J=5.80 Hz, 1H).

### Example 11

### N-[((5S)-3-{3-fluoro-4-[4-(6-{4-[(1E)-N-hydroxyethan-imidoyl]-phenyl}hex-5-ynoyl)piperazin-1-yl]phenyl}-2-oxo-1,3-oxazolidin-5-yl)methyl]-acetamide, HCl salt, 15.

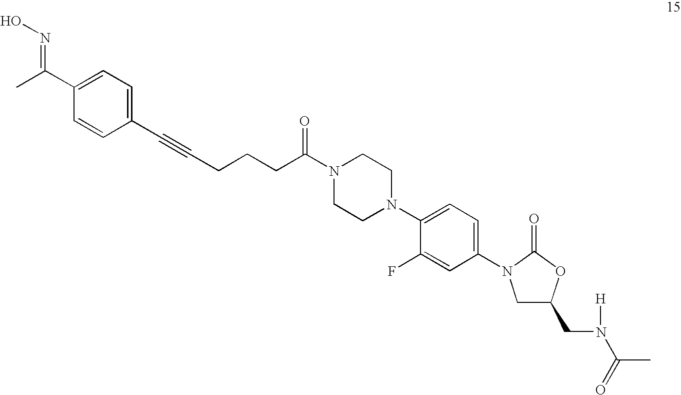

N-[((5S)-3(4-[4-(6-{4-acetylphenyl} hex-5-ynoyl)piperazine-1-yl]-3-fluorophenyl)-2-oxo-1,3-oxazolidin-5-yl)methyl]acetamide (220 mg, 0.40 mmol) was dissolved in 4 mL of a solution of 1:1 pyridine EtOH and treated with hydroxylamine.HCl (60 mg, 0.80 mmol). The reaction mixture was then heated to 50° C. for 2 hr at which time the reaction was complete. The reaction mixture was concentrated in vacuo and the organic product extracted with CH₂Cl₂. The combined organic layers were washed with 1 M HCl and brine and then dried over MgSO₄ and concentrated in vacuo to afford a white solid after trituration with Et₂O. ¹H NMR (300 MHz, DMSO-d₆) δ 11.31 (s, 1H), 8.24 (t, J=6 Hz, 1H), 7.63 (m, 2H), 7.50 (dd, J=3, 15 Hz, 1H), 7.40 (m, 2H), 7.17 (m, 1H), 7.05 (m, 1H), 4.70 (m, 1H), 4.08 (t, J=9 Hz, 1H), 3.70 (dd, J=6, 8 Hz, 1H), 3.62 (m, 4H), 3.40 (m, 2H), 3.37 (m, 4H), 2.94 (m, 4H), 2.13 (s, 3H), 1.83 (s, 3H), 1.80 (m, 2H).

### Example 12

### N-({(5S)-3-[4-(1-{6-[3-(aminomethyl)phenyl]hex-5-ynoyl}-3-methylazetidin-3-yl)phenyl]-2-oxo-1,3-oxazolidin-5-yl}methyl)propan-amide, 16.

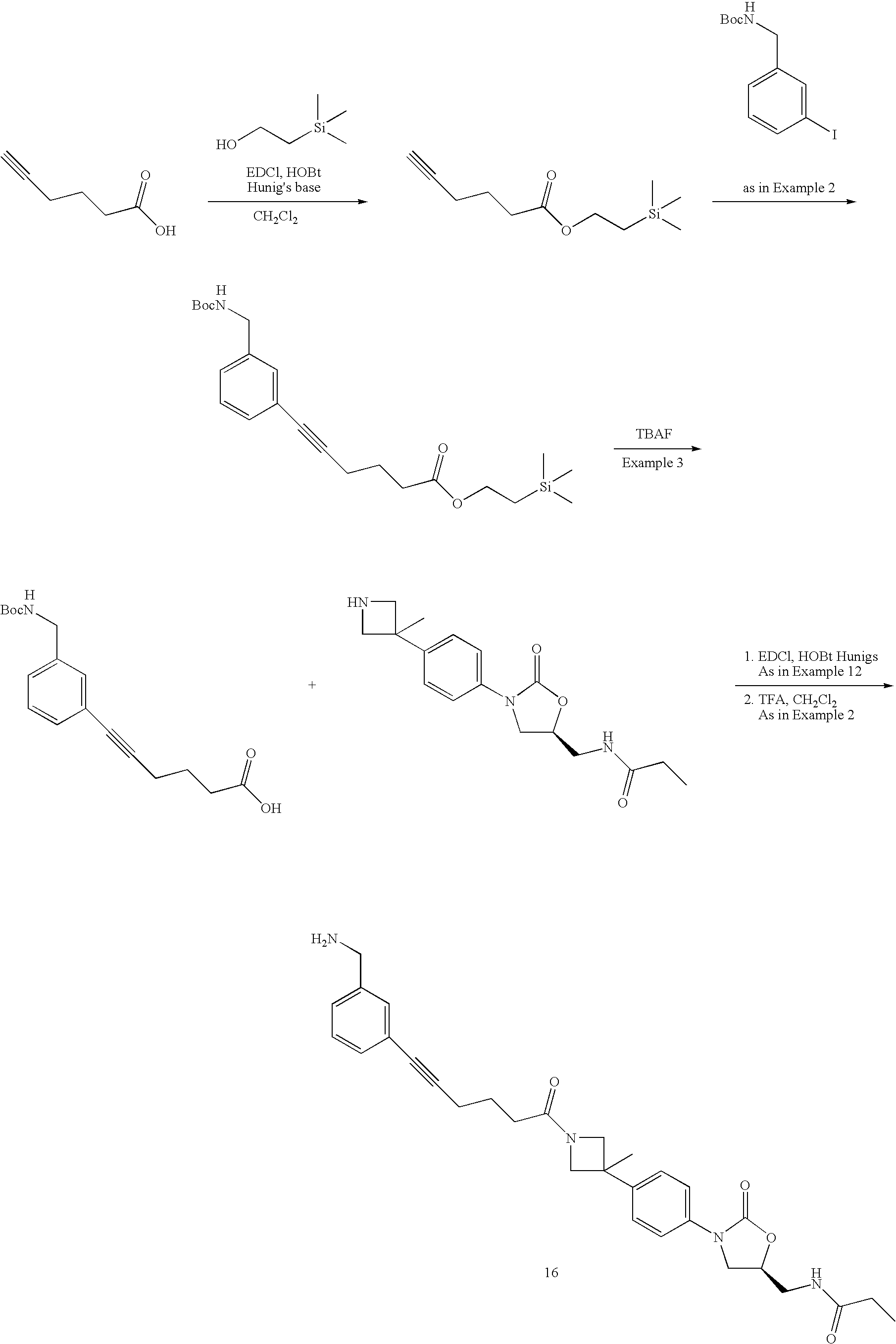

Prepared according to the following general procedure.

Step 1. 5-Hexynoic acid (44.6 mmol) and the alcohol (53.5 mmol) were stirred together at 0° C. and then HOBt (68.9 mmol), EDCI (53.5 mmol), and Hunig's base (133.8 mmol) were added successively. The ice bath was allowed to expire and the reaction warmed to rt and stirred 18 hr. After this period of time, the reaction mixture was poured into water and the organic product extracted with CH₂Cl₂; the combined organic layers were washed with 1M HCl, saturated aqueous NaHCO₃, water, and brine and then dried over MgSO₄ and filtered. The concentrated product was purified by chromatography to afford a clear oil. ¹H NMR (400 MHz, DMSO-d₆) δ 4.08 (t, J=8 Hz, 2H), 2.79(t, J=2 Hz, 1H), 2.34(t, J=4 Hz, 2H), 2.17(dt, J=4, 8 Hz, 2H), 1.66 (m, 2H), 0.92 (m, 2H), −0.01 (s, 9H).

Step 2. The alkyne was coupled with N-Boc(3-iodo)benzyl amine according to the procedure outlined in Example 2. ¹H NMR (400 MHz, DMSO-d₆) δ 7.39 (m, 1H), 7.22 (m, 4H), 4.08 (m, 2H), 2.43 (m, 2H), 1.77 (m, 2H), 1.37 (s, 9H), 1.29 (m, 4H), 0.92 (m, 2H), −0.03 (s, 9H).

Step 3. The silyl protecting group was removed with TBAF according to the general procedure outlines in Example 3. ¹H NMR (300 MHz, DMSO-d₆) δ 12.1 (s, 1H), 7.41 (t, J=9 Hz, 1H), 7.25 (m, 4H), 4.10 (d, J=6 Hz, 2H), 2.45 (t, J=6 Hz, 2H), 2.38 (t, J=9 Hz, 2H), 1.76 (m, 2H), 1.39 (s, 9H).

N-({(5S)-3-[4-(3-methylazetidin-3-yl)phenyl]-2-oxo-1,3-oxazolidin-5-yl}methyl)propanamide was prepared by the following method:

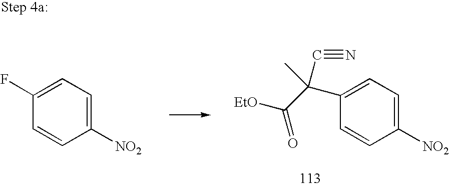

A suspension of anhydrous K₂CO₃ (13.0 g, 94.3 mmol) in CH₃CN (175 mL) is treated with ethyl cyanoacetate (5.0 mL, 47.1 mmol) at rt with stirring. The reaction is warmed to 75° C. for 20 min, then cooled to 0° C. with an ice bath followed by dropwise treatment with 1-fluoro-4-nitrobenzene (5.0 mL, 47.1 mmol) over 5 min. The ice bath is removed and the red suspension is warmed to 75° C. for 18 h. The dark red suspension is then cooled to rt, treated with iodomethane (26.4 mL, 424.2 mmol), K₂CO₃ (19.5 g, 141.4 mmol), acetone (60 mL), and then warmed to 60° C. for 24 h. The pink suspension is then cooled to rt and filtered through Celite (repeated EtOAc washings). After concentrating the filtrate in vacuo, the orange-brown residue is diluted with H₂O (300 mL) and extracted with EtOAc (2×200 mL). The combined organic extracts are washed with brine (150 mL), dried over MgSO₄, and concentrated in vacuo. Purification of the crude product via Biotage chromatography (eluting with 20% EtOAc/Hexane) affords 113 (4.87 g) as a white solid. ¹H NMR (400 MHz, CDCl₃) δ 8.27 (d, J=8.9 Hz, 2H), 7.73 (d, J=9.1 Hz, 2H), 4.26 (m, 2H), 1.99 (s, 3H), 1.26 (t, J=7.2 Hz, 3H).

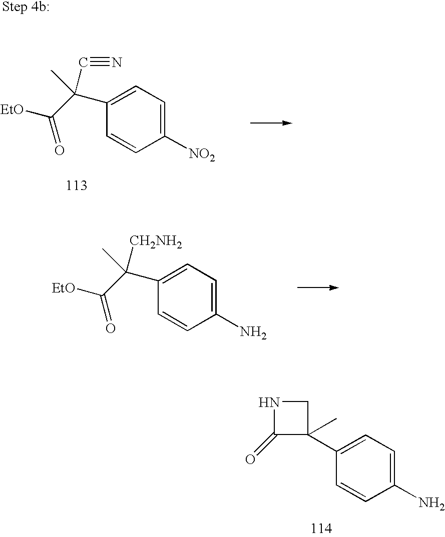

A solution 113 (15.8 g, 63.8 mmol) in absolute EtOH (650 mL) is treated with Raney nickel (34.5 g of a 50% slurry in H₂O) and subjected to hydrogenation in a Parr apparatus for 18 h (25-30 psi H₂, rt). The reaction mixture is then filtered through Celite (repeated EtOH washings) and concentrated in vacuo. Purification of the crude product via Biotage chromatography (eluting with 15% MeOH/EtOAc) affords the reduced amino-aniline compound corresponding to 113 (11.36 g) as a pale yellow oil:

¹H NMR (400 MHz, CDCl₃) δ 7.04 (d, J=8.7 Hz, 2H), 6.63 (d, J=8.7 Hz, 2H), 4.13 (m, 2H), 3.09 (d, J=13.3 Hz, 1H), 2.94 (d, J=13.5 Hz, 1H), 1.52 (s, 3H), 1.19 (t, J=7.2 Hz, 3H); ¹³C NMR (100 MHz, CDCl₃) δ 175.87, 145.17, 131.43, 127.06, 115.02, 60.61, 51.74, 51.14, 20.81, 14.05. A solution of this amino-aniline (8.3 g, 37.3 mmol) in THF (207 mL) is added dropwise to a 0° C. solution of methyl magnesium bromide (62.2 mL, 3.0 M in Et₂O) in THF (415 mL). When addition is complete, THF (30 mL) is used to rinse the addition funnel, and then the ice bath is removed and reaction stirred at rt for 3 h. The reaction contents are poured into saturated aqueous NH₄Cl (1000 mL) and volatiles are removed in vacuo. The aqueous phase is extracted with CHCl₃ (4×300 mL). The combined organic extracts are washed with H₂O (200 mL), brine (200 mL), dried over MgSO₄, and concentrated in vacuo. Purification of the crude product via Biotage chromatography (eluting with 50% EtOAc/hexane) affords 114 (4.65 g) as an off-white solid in 71% yield; ¹H NMR (300 MHz, CDCl₃) δ 7.17 (d, J=8.7 Hz, 2H), 6.65 (d, J=8.3 Hz, 2H), 6.13 (s, 1H), 3.48 (d, J=5.3 Hz, 1H), 3.36 (d, J=5.3 Hz, 1H), 1.62 (s, 3H); ¹³C NMR (100 MHz, CDCl₃) δ 173.44, 145.30, 130.60, 126.71, 115.15, 59.41, 51.40, 23.14; MS (ESI+) for C₁₀H₁₂N₂O m/z 177.1 (M+H)⁺.

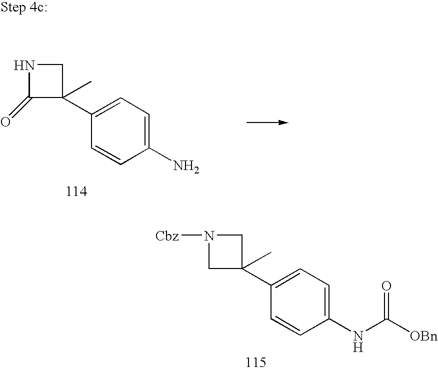

A solution of LiAlH₄ (79.2 mL, 1.0 M in THF) is diluted with THF (77 mL) and cooled to 0° C. To this is added a solution of 114 (4.65 g, 26.4 mmol) in THF (116 mL), with gas evolution. The ice bath is removed and the reaction was heated to reflux (75° C. oil bath) for 22 h, during which time the reaction became a white suspension. After cooling to rt, the mixture was treated successively with H₂O (3.01 mL), 5 N aqueous NaOH (2.71 mL), and H₂O (10.52 mL). The resulting suspension was diluted with EtOAc (600 mL), filtered through a pad of Celite, rinsing the Celite with additional EtOAc (400 mL). Concentration in vacuo affords the desired azetidine (4.76 g) as a yellow oil. A solution of this crude intermediate in acetone (85 mL) and H₂O (47 mL) was treated with sodium bicarbonate (19.96 g, 237.6 mmol). The resulting suspension was cooled to 0° C. and treated with benzylchloroformate (18.84 μL, 132.0 mmol) with gas evolution. The ice bath was removed and the reaction mixture was stirred overnight at rt. In the a.m., the reaction mixture was diluted with saturated aqueous sodium bicarbonate (200 mL) and extracted with EtOAc (3×200 mL). The combined organic extracts were washed with H₂O (100 mL), brine (100 mL), dried over MgSO₄, and concentrated in vacuo. Purification of the crude product via Biotage chromatography (eluting with 25% EtOAc/hexane) affords (4.75 g) as a pale yellow syrup; ¹H NMR (400 MHz, CDCl₃) δ 7.41-7.29 (m, 12H), 7.13 (d, J=8.7 Hz, 2H), 6.77 (s, 1H), 5.19 (s, 2H), 5.10 (s, 2H), 4.22 (d, J=8.3 Hz, 2H), 3.97 (d, J=8.3 Hz, 2H), 1.59 (s, 3H); MS (ESI—) for C₂₆H₂₆N₂O₄ m/z 429.0 (M−H)⁻.

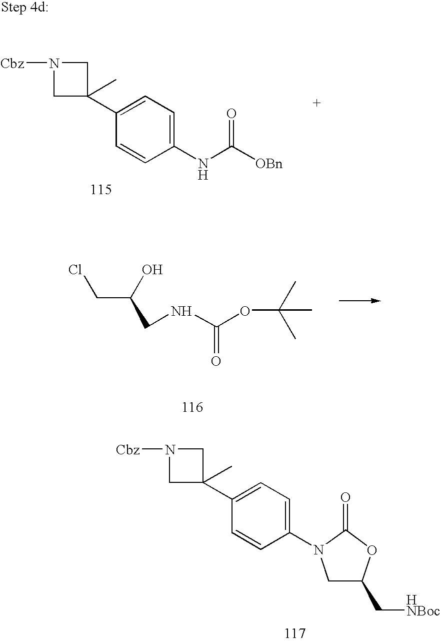

A solution of 116 (4.75 g, 11.03 mmol) in DMF (20 mL) at 0° C. is treated with LiOt-Bu (33.1 mL, 1.0 M solution in hexanes) dropwise over 25 min. After an additional 20 min, the reaction is treated with 116 (4.63 g, 22.07 mmol, U.S. Patent Publication Application 2002/0086900) and stirred 14 h with ice bath expiring. The reaction is quenched with saturated aqueous NH₄Cl (85 mL) and then extracted with EtOAc (3×80 mL). The combined organic extracts are washed with brine, dried over MgSO₄, and concentrated in vacuo. Purification of the crude product via Biotage chromatography (eluting with 50% EtOAc/hexane) affords 117 (4.16 g) as a white solid; ¹H NMR (400 MHz, DMSO-d₆) δ 7.50 (d, J=8.7 Hz, 2H), 7.39-7.30 (m, 5H), 7.31 (d, J=8.7 Hz, 2H), 7.22 (t, J=5.9 Hz, 1H), 5.05 (s, 2H), 4.68 (m, 1H), 4.14 (m, 2H), 4.10 (t, J=9.0 Hz, 1H), 3.95 (m, 2H), 3.78 (dd, J=9.0, 5.9 Hz, 1H), 3.27 (t, J=5.4 Hz, 2H), 1.54 (s, 3H), 1.35 (s, 9H); ¹³C NMR (100 MHz, DMSO-d₆) δ 155.86, 154.03, 141.54, 136.73, 136.56, 128.28, 127.75, 127.52, 125.73, 117.96, 77.96, 71.28, 65.62, 47.03, 42.79, 37.69, 28.79, 28.00.

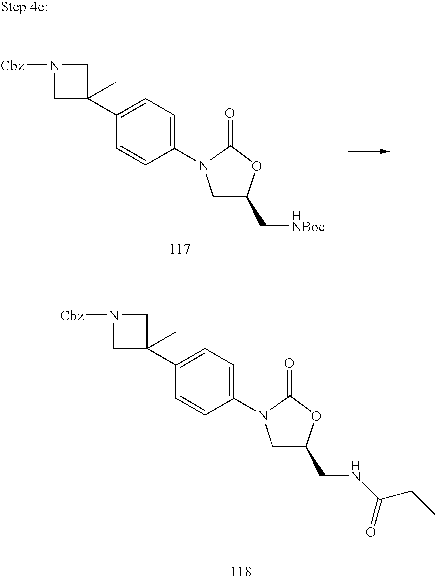

A solution of 117 (4.10 g, 8.37 mmol) in MeOH (20 mL) at 0° C. is treated with 4 M HCl-dioxane (5.0 mL) and stirred for 10 min. The ice bath is then removed, and after stirring 20 h at rt, the solution is concentrated in vacuo. The resulting pale yellow solid is subsequently treated with pyridine (12 mL) and propionic anhydride (6 mL) in CH₂Cl₂ (30 mL) at 0° C. and stirred 14 h with bath expiring. The reaction is quenched with H₂O (150 mL), and the layers are separated. The organic layer is washed with 1.0 M HCl (2×50 mL), brine (50 mL), dried over MgSO₄, and concentrated in vacuo. Trituration (Et₂O) affords (2.98 g) as a white solid in 79% yield; ¹H NMR (400 MHz, DMSO-d₆) δ8.18 (t, J=5.8 Hz, 1H), 7.50 (d, J=8.7 Hz, 2H), 7.39-7.30 (m, 5H), 7.31 (d, J=8.5 Hz, 2H), 5.05 (s, 2H), 4.72 (m, 1H), 4.13 (m, 2H), 4.11 (t, J=8.9 Hz, 1H), 3.96 (m, 2H), 3.74 (dd, J=8.9, 6.2 Hz, 1H), 3.42 (m, 2H), 2.10 (q, J=7.7 Hz, 2H), 1.54 (s, 3H), 0.96 (t, J=7.7 Hz, 3H); ¹³C NMR (100 MHz, DMSO-d₆) δ 173.65, 155.88, 154.04, 141.56, 136.74, 136.52, 128.29, 127.75, 127.53, 125.75, 117.98, 71.43, 65.64, 47.11, 41.24, 37.69, 28.78, 28.25, 9.81.

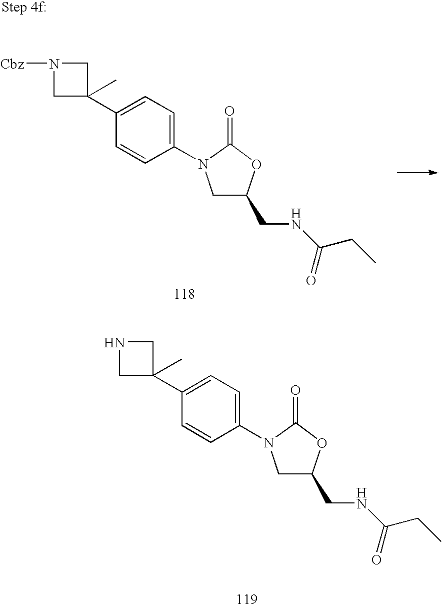

A solution of 118 (2.78 g, 6.16 mmol) in 5:1 MeOH:THF (60 nL) was placed under N₂ (g) and then treated with 10% Pd/C (278 mg). The reaction vessel was then charged with H₂ (g) and the reaction mixture was stirred overnight at rt. In the a.m., the mixture was filtered and concentrated in vacuo. Purification of the crude product via Biotage chromatography (eluting first with 10% MeOH/CH₂Cl₂ and then with 5-15% MeOH(NH₃)/CH₂Cl₂) affords (1.79 g) as a white solid; ¹H NMR (400 MHz, DMSO-d₆) δ 8.18 (t, J=5.8 Hz, 1H), 7.47 (d, J=8.9 Hz, 2H), 7.21 (d, J=8.7 Hz, 2H), 4.71 (m, 1H), 4.10 (t, J=9.0 Hz, 1H), 3.74 (t, J=6.4 Hz, 1H), 3.73 (d, J=6.6 Hz, 2H), 3.42 (m, 2H), 3.38 (d,J=7.5 Hz, 2H), 2.10 (q, J=7.5 Hz, 2H), 1.53 (s, 3H), 0.96 (t, J=7.6 Hz, 3H); ¹³C NMR (100 MHz, DMSO-d₆) δ 173.65, 154.06, 144.21, 135.92, 125.29, 117.95, 71.39, 58.27, 47.15, 41.91, 41.25, 28.64, 28.25, 9.82 1.41.

Step 5. The amide bond forming reaction with the azetidine nitrogen was performed according to the general procedure outlined in Example 12, step 1. ¹H NMR (400 MHz, DMSO-d₆) δ 8.17 (t, J=4 Hz, 1H), 7.49 (d, J=8 Hz, 2H), 7.41 (t, J=6 Hz, 1H), 7.30 (d, J=8 Hz, 2H), 7.26-7.18 (m, 4H), 4.72 (m, 1H), 4.33 (d, J=8 Hz, 1H), 4.13 (d, J=8 Hz, 1H), 4.09 (m, 3H), 4.04 (d, J=8 Hz, 1H), 3.86 (d, J=8 Hz, 1H), 3.74 (dd, J=4, 6 Hz, 1H), 3.42 (m, 2H), 2.44 (t, J=8 Hz, 2H), 2.24 (dt, J=8, 4 Hz, 2H), 2.09 (q, J=8 Hz, 2H), 1.75 (m, 2H), 1.53 (s, 3H), 1.38 (s, 9H), 0.95 (t, J=8 Hz, 3H).

Step 6. The Boc protecting group was removed as outline in Example 2 to afford the desire product. ¹H NMR (400 MHz, DMSO-d₆) δ 8.19 (t, J=8 Hz, 1H), 7.50 (d, J=8 Hz, 2H), 7.40 (s, 1H), 7.30 (d, J=8 Hz, 2H), 7.26 (m, 3H), 4.72 (m, 1H), 7.32 (d, J=8 Hz, 1H), 4.13 (m, 3H), 3.86 (d, J=8 Hz, 1H), 3.74 (s, 3H), 3.60 (s, 2H), 3.43 (m, 2H), 2.44 (t, J=8 Hz, 2H), 2.25 (t, J=8 Hz, 2H), 2.09 (q, J=8 Hz, 2H), 1.76 (m, 2H), 1.54 (s, 3H), 0.96 (t, J=8 Hz, 3H).

### Example 13

### N-{[(5S)-3-(4-{4-[6-(4-acetylphenyl)hex-5-ynoyl]piperazin-1-yl}-3-fluorophenyl)-2-oxo-1,3-oxazolidin-5-yl]methyl}acetamide, 17.

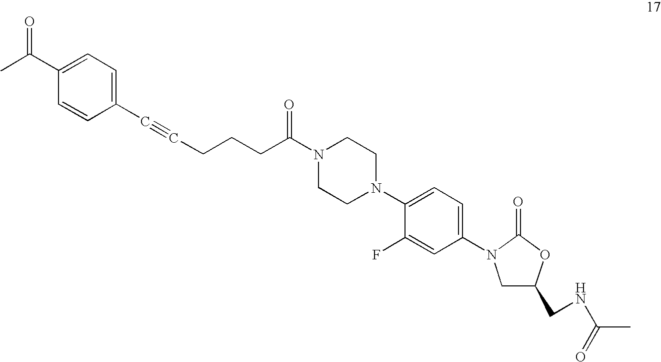

Prepared as in Example 2, step 1, with nonessential modifications but substituting the appropriate reactants, (4-iodo)acetophenone was used in the Sonogashira coupling reaction. ¹H NMR (400 MHz, DMSO-d₆) δ 8.24 (t, J=8 Hz, 1H), 7.92 (d, J=8 Hz, 2H), 7.54 (d, J=8 Hz, 2H), 7.50 (dd, J=16, 4 Hz, 1H), 7.17 (m, 1H), 7.05 (dd, J=12, 12 Hz, 1H), 4.71 (m, 1H), 4.08 (t, J=8 Hz, 1H), 3.70 (dd, J=8, 10 Hz, 1H), 3.62 (m, 4H), 3.40 (t, J=4 Hz, 2H), 2.93 (m, 4H), 2.57 (s, 3H), 2.53 (m, 4H), 1.83 (s, 3H), 1.82 (m, 2H).

### Example 14

### N-({(5S)-3-[3-fluoro-4-(4-{6-[4-(1H-imidazol-1-ylmethyl)phenyl]hex-5-ynoyl}piperazin-1-yl)phenyl]-2-oxo-1,3-oxazolidin-5-yl}methyl)acetamide, 18.

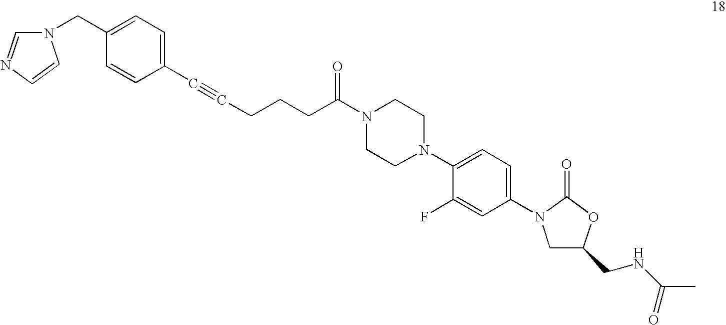

Prepared as in Example 2, step 1, with nonessential modifications but substituting the appropriate reactants. The pyrazolylmethyl-iodo-benzene was obtained by alkylation of pyrrazole with 4-iodobenzyl bromide according to the literature procedure (OPPI, 2000, 32(4), 385). ¹H NMR (400 MHz, DMSO-d₆) δ 8.25 (m, 1H), 7.81 (m, 1H), 7.50 (dd, J=3, 15 Hz, 1H), 7.38 (d, J=9 Hz, 2H), 7.20 (d, J=9 Hz, 2H), 7.15(m, 1H), 7.05 (m, 1H), 6.94 (m, 1H), 5.20 (s, 2H), 4.70 (m, 1H), 4.08 (dd, J=9, 9 Hz, 1H), 3.70 (dd, J=6, 9 Hz, 1H), 3.61 (m, 4H), 3.40 (t, J=6 Hz, 2H), 2.94 (m, 4H), 2.47 (m, 4H), 1.83 (s, 3H), 1.78 (m, 2H).

### Example 15

### N-[((5S)-3-{3-fluoro-4-[4-(6-{4-[(methylamino)methyl]phenyl}hex-5-ynoyl)piperazin-1-yl]phenyl}-2-oxo-1,3-oxazolidin-5-yl)methyl]acetamide, 19.

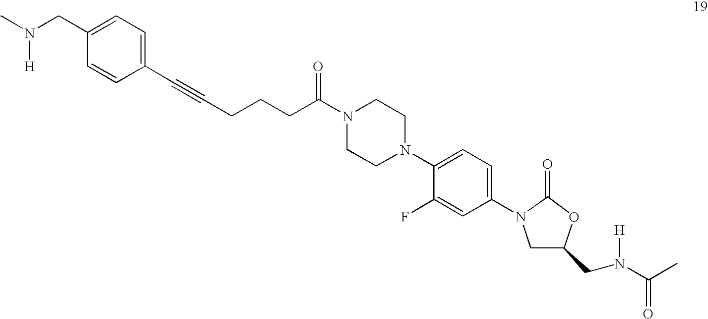

Prepared according to the steps outlined in Example 12 with nonessential modifications but substituting the appropriate reactants N-methyl-N-BOC-4-iodo benzylamine was used in the Sonogashira coupling reaction. ¹H NMR (400 MHz, DMSO-d₆) 68.24 (t, J=8 Hz, 1H), 7.50 (dd, J=4, 12 Hz, 1H), 7.36 (s, 1H), 7.28 (m, 4H), 7.17 (dd, J=4, 12 Hz, 1H), 7.05 (t, J=12 Hz, 1H), 4.71 (m, 1H), 4.08 (t, J=8 Hz, 1H), 3.70 (dd, J=8, 10 Hz, 1H), 3.64 (m, 4H), 3.40 (t, J=4 Hz, 2H), 3.32 (bs, 1H), 2.94 (m, 5H), 2.47 (m, 4H), 2.25 (s, 3H), 1.83 (s, 3H), 1.79 (m, 2H).

### Example 16

### N-{[(5S)-3-(4-{4-[5-(4-aminophenyl)pent-4-ynoyl]piperazin-1-yl}-3-fluorophenyl)-2-oxo-1,3-oxazolidin-5-yl]methyl}acetamide, 20.

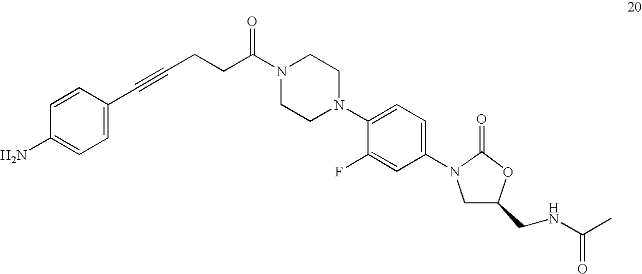

Prepared as in Example 2, step 1, with nonessential modifications but substituting the appropriate reactants; ¹H NMR (400 MHz, DMSO-d₆) δ 8.25 (m, 1H), 7.51 (dd, J=4, 16 Hz, 1H), 7.16 (dd, J=2, 4 Hz, 1H), 7.08 (m, 1H), 7.02 (d, J=12 Hz, 2H), 6.47 (d, J=8 Hz, 2H), 5.37 (s, 2H), 4.71 (m, 1H), 4.08 (t, J=8 Hz, 1H), 3.70 (dd, J=8, 16 Hz, 1H), 3.63 (m, 4H), 3.40 (m, 2H), 2.93 (m, 4H), 2.60 (m, 4H), 1.83 (s, 3H).

### Example 17

### methyl 4-{5-[4-(4-{(5S)-5-[(acetylamino)methyl]-2-oxo-1,3-oxazolidin-3-yl}-2-fluorophenyl)piperazin-1-yl]-5-oxopent-1-ynyl}-L-phenylalaninate, 21.

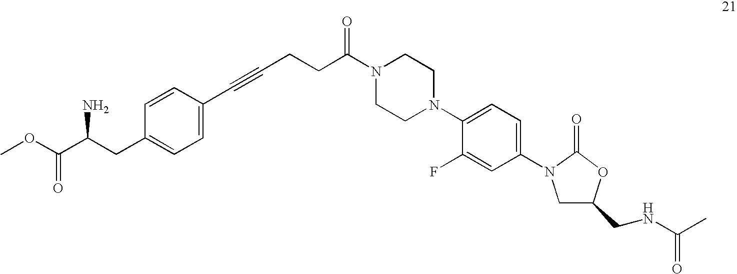

Prepared from commercially available N-(tert-butoxycarbonyl)-4-iodo-L-phenylalanine by Sonogashira coupling and subsequent removal of the Boc protecting group with TFA as in Example 2. ¹H NMR (400 MHz, DMSO-d₆) δ 8.25 (m, 1H), 7.50 (dd, J=3, 15 Hz, 1H), 7.27 (d, J=9 Hz, 2H), 7.19 (m, 1H), 7.14 (d, J=9 Hz, 2H), 7.03 (m, 1H), 4.71 (m, 1H), 4.08 (dd, J=9, 9 Hz, 1H), 3.69 (dd, J=6, 9 Hz, 2H), 3.63 (m, 4H), 3.56 (s, 3H), 3.40 (m, 2H), 2.94 (m, 4H), 2.82 (dd, J=6, 12 Hz, 2H), 2.67 (m, 4H), 2.66 (m, 2H), 1.83 (s, 3H).

### Example 18

### N-{[(5S)-3-(4-{4-[5-(2,4-dioxo-1,2,3,4-tetrahydropyrimidin-5-yl)pent-4-ynoyl]piperazin-1-yl}-3-fluorophenyl)-2-oxo-1,3-oxazolidin-5-yl]methyl}acetamide, 22.

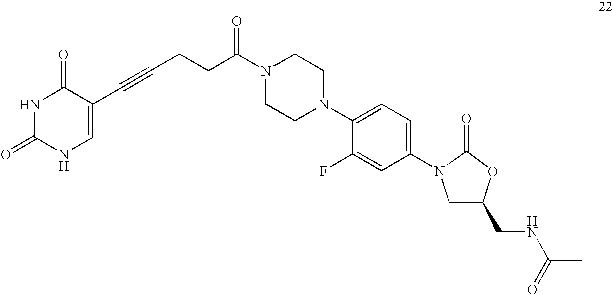

Prepared from the iodouracil by Sonogashira coupling as in Example 2, step 1. ¹H NMR (400 MHz, DMSO-d₆) δ 11.31 (s, 1H), 11.15 (s, 1H), 8.25 (t, J=4 Hz, 1H), 7.65 (d, J=4 Hz, 1H), 7.50 (dd, J=4, 16 Hz, 1H), 7.16 (dd, J=2, 8 Hz, 1H), 7.05 (t, J=8 Hz, 1H), 4.71 (m, 1H), 4.08 (t, J=8 Hz, 1H), 3.70 (dd, J=8, 10 Hz, 1H), 3.62 (m, 4H), 3.40 (t, J=4 Hz, 2H), 2.95 (m, 4H), 2.61 (m, 4H), 1.83 (s, 3H).

### Example 19

### N-({(5S)-3-[4-(4-{7-[4-(aminomethyl)phenyl]hept-6-ynoyl}piperazin-1-yl)-3-fluorophenyl]-2-oxo-1,3-oxazolidin-5-yl}methyl)acetamide, 23.

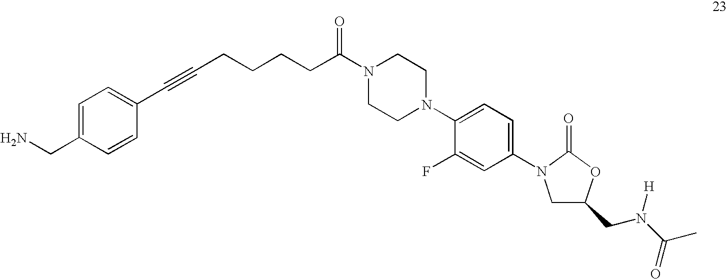

Prepared as in Example 2 with nonessential modifications but substituting the appropriate reactants; ¹H NMR (300 MHz, DMSO-d₆) δ 8.21 (m, 1H), 7.46 (m, 1H), 7.29 (m, 4H), 7.15 (m, 1H), 7.00 (m, 1H), 4.05 (m, 2H), 3.67 (m, 2H), 3.57 (m, 4H), 3.37 (m, 3H), 2.90 (m, 4H), 2.40 (m, 4H), 1.80 (s, 3H), 1.63 (m, 2H), 1.54 (m, 2H).

### Example 20

### N-{[(5S)-3-(3-fluoro-4-{4-[7-(4-hydroxyphenyl)hept-6-ynoyl]piperazin-1-yl}phenyl)-2-oxo-1,3-oxazolidin-5-yl]methyl}acetamide, 24.

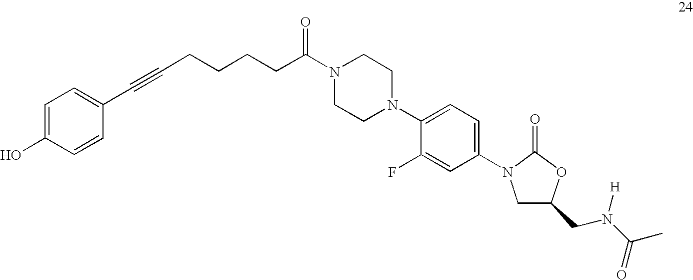

Prepared as in Example 3 with nonessential modifications but substituting the appropriate reactants; ¹H NMR (300 MHz, DMSO-d₆) δ 9.71 (s, 1H), 8.24 (m, 1H), 7.50 (dd, J=3, 9 Hz, 1H), 7.18 (d, J=9 Hz, 2H), 7.14 (m, 1H), 7.03 (dd, J=9, 9 Hz, 1H), 6.70 (d, J=9 Hz, 2H), 4.70 (m, 1H), 4.08 (t, J=9 Hz, 1H), 3.70 (dd, J=6, 9 Hz, 1H), 3.60 (m, 4H), 3.40 (t, J=6 Hz, 2H), 2.92 (m, 4H), 2.40 (m, 4H), 1.83 (s, 3H), 1.65 (m, 2H), 1.57 (m, 2H).

### Example 21

### N-{[(5S)-3-(3-fluoro-4-{4-[7-(3-hydroxyphenyl)hept-6-ynoyl]piperazin-1-yl}phenyl)-2-oxo-1,3-oxazolidin-5-yl]methyl}acetamide, 25.

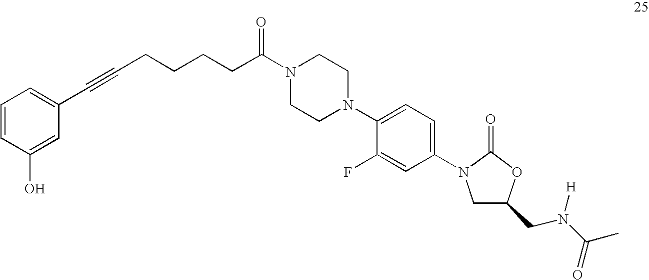

Prepared as in Example 3 with nonessential modifications but substituting the appropriate reactants; ¹H NMR (300 MHz, DMSO-d₆) δ 9.52 (s, 1H), 8.21 (m, 1H), 7.46 (dd, J=4, 16 Hz, 1H), 7.12 (dd, J=4, 8 Hz, 1H), 7.08 (m, 1H), 7.00 (dd, J=12, 12 Hz, 1H), 6.75 (d, J=8 Hz, 1H), 6.68 (m, 2H), 4.67 (m, 1H), 4.04 (t, J=8 Hz, 1H), 3.66 (dt, J=8, 12 Hz, 1H), 3.57 (m, 4H), 3.36 (t, J=8 Hz, 2H), 2.88 (m, 4H), 2.39 (m, 4H), 1.79 (s, 3H), 1.62 (m, 2H), 1.53 (m, 2H).

### Example 22

### N-({(5S)-3-[4-(4-{6-[4-(aminomethyl)phenyl]hex-5-ynoyl}piperazin-1-yl)-3-fluorophenyl]-2-oxo-1,3-oxazolidin-5-yl}methyl)acetamide, 26.

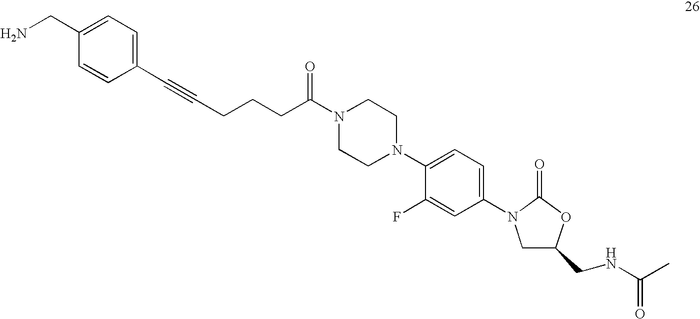

Prepared as in Example 2 with nonessential modifications but substituting the appropriate reactants; ¹H NMR (300 MHz, DMSO-d₆) δ 8.24 (m, 1H), 7.50 (dd, J=3, 15 Hz, 1H), 7.32 (m, 4H), 7.17 (dd, J=2, 9 Hz, 1H), 7.05 (m, 1H), 4.71 (m, 1H), 4.08 (t, J=9 Hz, 1H), 3.71 (m, 3H), 3.62 (m, 4H), 3.40 (m, 2H), 3.30 (bs, 2H), 2.96 (m, 4H), 2.46 (m, 4H), 1.83 (s, 3H), 1.79 (m, 2H).

### Example 23

### N-{[(5S)-3-(3-fluoro-4-{4-[6-(3-hydroxyphenyl)hex-5-ynoyl]piperazin-1-yl}phenyl)-2-oxo-1,3-oxazolidin-5-yl]methyl}acetamide, 27.

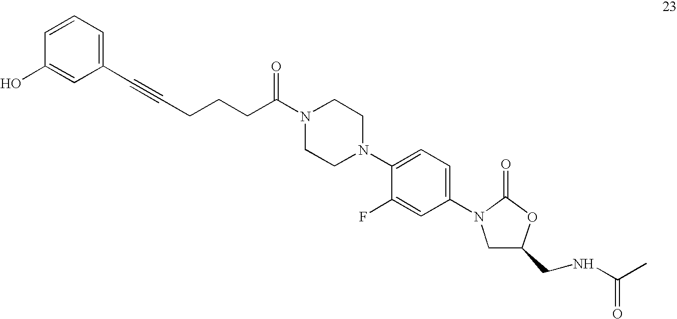

Prepared as in Example 3 with nonessential modifications but substituting the appropriate reactants; ¹H NMR (300 MHz, DMSO-d₆) δ 9.56 (s, J=1 Hz, 1H), 8.24 (m, 1H), 7.50 (dd, J=4, 8 Hz, 1H), 7.16 (dd, J=4, 12 Hz, 1H), 7.12 (m, 1H), 7.03 (dd, J=8, 8 Hz, 1H), 6.78 (m, 1H), 6.73 (m, 2H), 4.71 (m, 1H), 4.08 (t, J=8 Hz, 1H), 3.70 (dd, J=8, 12 Hz, 1H), 3.60 (m, 4H), 3.40 (t, J=4 Hz, 2H), 2.92 (m, 4H), 2.41 (m, 2H), 1.83 (s, 3H), 1.65 (m, 2H), 1.57 (m, 2H).

### Example 24

### N-({(5S)-3-[4-(4-{5-[4-(aminomethyl)phenyl]pent-4-ynoyl}piperazin-1-yl)-3-fluorophenyl]-2-oxo-1,3-oxazolidin-5-yl}methyl)acetamide, 28.

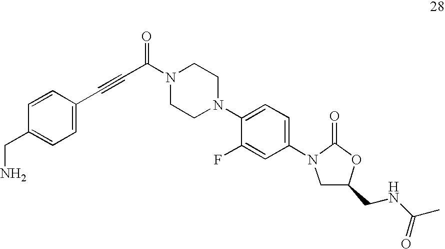

Prepared as in Example 2 with nonessential modifications but substituting the appropriate reactants; ¹H NMR (300 MHz, DMSO-d₆) δ 8.24 (m, 1H), 7.50 (dd, J=2, 12 Hz, 1H), 7.30 (m, 4H), 7.17 (m, 1H), 7.02 (m, 1H), 4.70 (m, 1H), 4.08 (m, 1H), 3.71 (m, 3H), 3.63 (m, 4H), 3.40 (t, J=6 Hz, 2H), 2.95 (m, 4H), 2.67 (m, 4H), 2.65 (m, 2H), 1.83 (s, 3H).

### Example 25

### N-{[(5S)-3-(3-fluoro-4-{4-[5-(3-hydroxyphenyl)pent-4-ynoyl]piperazin-1-yl}phenyl)-2-oxo-1,3-oxazolidin-5-yl]methyl}acetamide, 29.

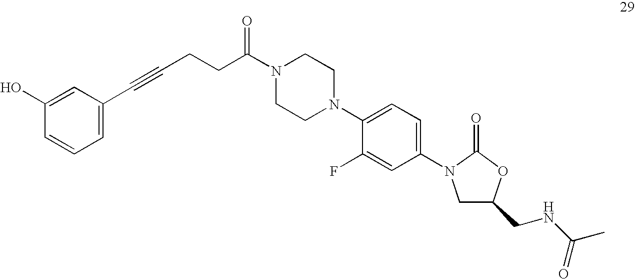

Prepared as in Example 3 with nonessential modifications but substituting the appropriate reactants; ¹H NMR (300 MHz, DMSO-d₆) δ 9.57 (s, 1H), 8.24 (m, 1H), 7.49 (dd, J=4, 16 Hz, 1H), 7.17 (dd, J=1, 8 Hz, 1H), 7.12 (dd, J=8, 8 Hz, 1H), 7.03 (t, J=8 Hz, 1H), 6.78 (d, J=8 Hz, 1H), 6.74 (m, 2H), 4.70 (m, 1H), 4.08 (m, 1H), 3.70 (dd, J=8, 12 Hz, 1H), 3.63 (m, 4H), 3.40 (m, 2H), 2.94 (m, 4H), 2.66 (m, 4H), 1.83 (s, 3H).

### Example 26

### N-{[(5S)-3-(3-fluoro-4-{4-[5-(4-hydroxyphenyl)pent-4-ynoyl]piperazin-1-yl}phenyl)-2-oxo-1,3-oxazolidin-5-yl]methyl}acetamide, 30.

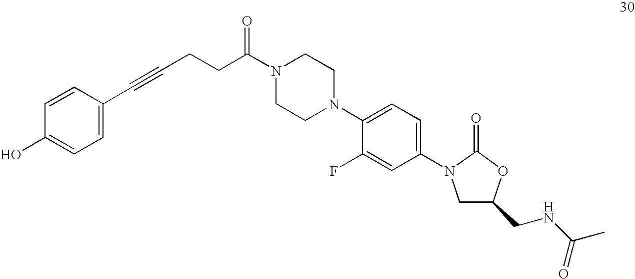

Prepared as in Example 3 with nonessential modifications but substituting the appropriate reactants; ¹H NMR (300 MHz, DMSO-d₆) δ 9.72 (s, 1H), 8.24 (m, 1H), 7.50 (dd, J=4, 16 Hz, 1H), 7.17 (m, 3H), 7.03 (dd, J=8, 8 Hz, 1H), 6.71 (d, J=8 Hz, 2H), 4.71 (m, 1H), 4.08 (dd, J=8, 8 Hz, 1H), 3.69 (dd, J=8, 12 Hz, 1H), 3.63 (m, 4H), 3.40 (t, J=4 Hz, 2H), 2.94 (m, 4H), 2.63 (m, 4H), 1.83 (s, 3H).

### Example 27

### N-({(5S)-3-[3-fluoro-4-(4-hept-6-ynoylpiperazin-1-yl)phenyl]-2-oxo-1,3-oxazolidin-5-yl}methyl)acetamide, 31.

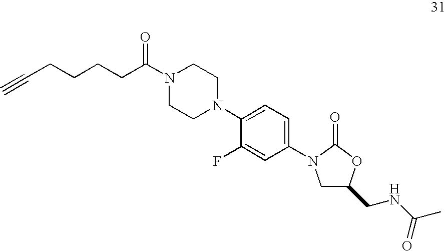

Prepared as in Example 1 with nonessential modifications but substituting the appropriate reactants; ¹H NMR (300 MHz, DMSO-d₆) δ 8.24 (m, 1H), 7.50 (dd, J=3, 15 Hz, 1H), 7.18 (dd, J=3, 9 Hz, 1H), 7.07 (dd, J=9, 9 Hz, 1H), 4.70 (m, 1H), 4.08 (m, 1H), 3.70 (dd, J=6, 9 Hz, 1H), 3.60 (m, 4H), 3.40 (m, 2H), 2.9 (m, 4H), 2.77 (m, 1H), 2.36 (m, 2H), 2.19 (dt, J=3, 6 Hz, 2H), 1.83 (s, 3H), 1.60 (m, 2H), 1.48 (m, 2H).

### Example 28

### N-({(5S)-3-[4-(4-{7-[3-(aminomethyl)phenyl]hept-6-ynoyl}-piperazin-1-yl)-3-fluorophenyl]-2-oxo-1,3-oxazolidin-5-yl}methyl)acetamide, 32.

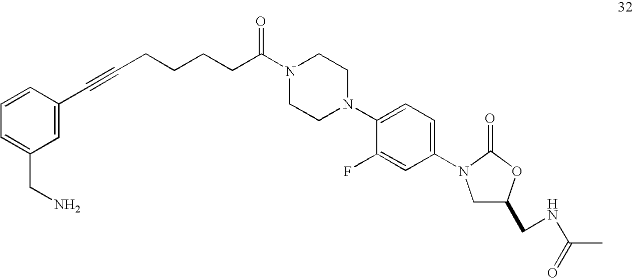

Prepared as in Example 2 with nonessential modifications but substituting the appropriate reactants; ¹H NMR (300 MHz, DMSO-d₆) δ 8.24 (m, 1H), 7.49 (dd, J=15, 2 Hz, 1H), 7.36 (bs, 1H), 7.27-7.15 (m, 4H), 7.03 (m, 1H), 4.70 (m, 1H), 4.08 (m, 1H), 3.70 (m, 2H), 3.68 (bs, 2H), 3.59 (m, 4H), 3.40 (m, 2H), 2.92 (m, 4H), 2.43 (m, 4H), 1.83 (s, 3H), 1.70-1.55 (m, 5H).

### Example 29

### N-({(5S)-3-[4-(4-{5-[3-(aminomethyl)phenyl]pent-4-ynoyl}piperazin-1-yl)-3-fluorophenyl]-2-oxo-1,3-oxazolidin-5-yl}-methyl)acetamide, 33.

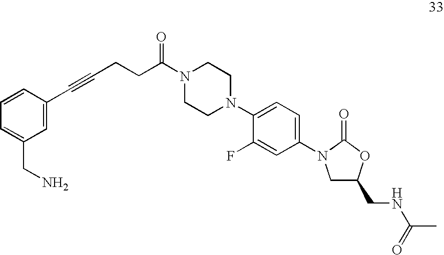

Prepared as in Example 2 with nonessential modifications but substituting the appropriate reactants; ¹H NMR (400 MHz, DMSO-d₆) δ 8.24 (m, 1H), 7.50 (dd, J=16, 4 Hz, 1H), 7.36 (m, 1H), 7.26 (m, 2H), 7.21-7.15 (m, 2H), 7.03 (dd, J=8, 8 Hz, 1H), 4.71 (m, 1H), 4.08 (dd, J=8 Hz, 1H), 3.68 (m, 3H), 3.64 (m, 4H), 3.40 (m, 2H), 2.95 (m, 4H), 2.67 (m, 4H), 1.83 (s, 3H), 1.22 (bs, 2H).

### Example 30

### N-({(5S)-3-[4-(4-{3-[3-(Aminomethyl)phenyl]prop-2-ynyl}-piperazin-1-yl)-3-fluorophen-yl]-2-oxo-1,3-oxazolidin-5-yl}methyl)acetamide, 34.

Step 1: tert-butyl 3-{3-[4-(4-{(5S)-5-[(acetylamino)methyl]-2-oxo-1,3-oxazolidin-3-yl}-2-fluorophenyl)piperazin-1-yl]prop-1-ynyl}benzylcarbamate, 34A

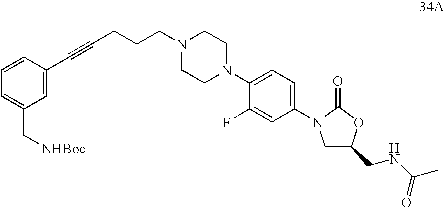

Prepared as in Example 2 with nonessential modifications but substituting the appropriate reactants; to afford the desired coupled product in 58-62% yield after Biotage chromatography with 3% MeOH/CH₂Cl₂; ¹H NMR (400 MHz, DMSO-d₆) δ 8.24 (m, 1H), 7.47 (dd, J=16, 4 Hz, 1H), 7.43 (m, 1H), 7.32 (m, 1H), 7.31 (s, 2H), 7.23 (m, 1H), 7.17 (dd, J=8, 2 Hz, 1H), 7.07 (dd, J=12, 12 Hz, 1H), 4.70 (m, 1H), 4.10 (m, 3H), 3.70 (dd, J=4, 8 Hz, 1H), 3.60 (s, 2H), 3.40 (m, 2H), 3.02 (m, 4H), 2.70 (m, 4H), 1.83 (s, 3H), 1.39 (s, 9H).

Step 2 N-({(5S)-3-[4-(4-{3-[3-(Aminomethyl)phenyl]prop-2-ynyl}piperazin-1-yl)-3-fluorophen-yl]-2-oxo-1,3-oxazolidin-5-yl}methyl)acetamide, 34.

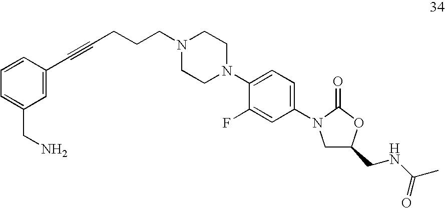

The NBoc (34A) amine was suspended in CH₂Cl₂ (ca. 10 mL) and treated with TFA (4 mL) at 0° C. for 30 minutes. TLC analysis revealed that the reaction was complete after 1 hr. The crude reaction mixture was concentrated in vacuo and subjected to Biotage chromatography with 5-6% MeOH/CH₂Cl₂/NH₃ to afford the desired free amine (4b, 0.452 g, 0.94 mmol) in 32% yield after trituration with Et₂O; ¹H NMR (300 MHz, DMSO-d₆) δ 8.24 (m, 1H), 7.48 (m, 2H), 7.30 (m, 3H), 7.17 (dd, J=9, 3 Hz, 1H), 7.08 (m, 1H), 4.70 (m, 1H), 4.08 (dd, J=9, 9 Hz, 1H), 3.70 (m, 3H), 3.59 (m, 2H), 3.40 (m, 2H), 3.03 (m, 4H), 2.70 (m, 4H), 2.24 (bs, 2H), 1.83 (s, 3H).

### Example 31

### Anti-Bacterial Activity

The in vitro MICs of test compounds were determined by a standard agar dilution method. A stock drug solution of each analog was prepared in the preferred solvent, usually DMSO: H₂O (1:3). Serial 2-fold dilutions of each sample are made using 1.0 ml aliquots of sterile distilled water. To each 1.0 ml aliquot of drug was added 9 ml of molten Mueller Hinton agar medium. The drug-supplemented agar was mixed, poured into 15×100 mm petri dishes, and allowed to solidify and dry prior to inoculation.

Vials of each of the test organisms are maintained frozen in the vapor phase of a liquid nitrogen freezer. Test cultures are grown overnight at 35° C. on the medium appropriate for the organism. Colonies are harvested with a sterile swab, and cell suspensions are prepared in Trypticase Soy broth (TSB) to equal the turbidity of a 0.5 McFarland standard. A 1:20 dilution of each suspension was made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the organism, was read and recorded.

The anti-bacterial activity for the example compounds is given in Table 1.

TABLE 1
Antibacterial Activity Minimum Inhibitory Concentration (μg/mL)

| Compound No. | SAUR 9213 MIC | SPNE 9912 MIC | HINF 30063 MIC |
|---|---|---|---|
| 4 | 4 | 1 | 64 |
| 6 | 2 | 1 | 16 |
| 7 | 4 | 1 | >64 |
| 8 | 4 | <0.5 | >32 |
| 9 | 4 | 2 | 16 |
| 10 | 4 | 2 | 32 |
| 11 | 4 | 2 | 16 |
| 12 | 16 | 2 | >64 |
| 13 | >64 | 64 | >64 |
| 14 | 8 | 2 | >64 |
| 15 | >16 | 2 | >16 |
| 16 | 16 | 2 | 16 |
| 17 | >64 | 2 | >64 |
| 18 | 16 | 2 | >64 |
| 19 | 4 | 0.25 | 32 |
| 20 | 4 | 2 | 64 |
| 21 | 32 | 4 | >64 |
| 22 | 32 | 4 | 32 |
| 23 | 4 | 0.25 | 32 |
| 24 | 8 | 2 | >64 |
| 25 | 8 | 2 | >64 |
| 26 | 4 | 0.25 | 16 |
| 27 | 8 | 1 | >64 |
| 28 | 4 | 0.5 | 16 |
| 29 | 4 | 1 | >64 |
| 30 | 4 | 1 | 64 |
| 31 | 4 | 1 | 64 |
| 32 | 4 | 2 | 64 |
| 33 | 4 | 1 | 16 |
| 35 | 64 | 16 | >64 |
| 36 | 4 | 0.5 | >64 |

What is claimed is:

1. A compound of formula I 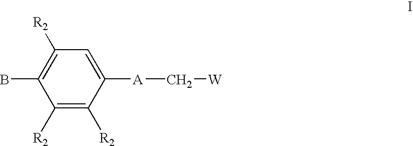
or pharmaceutically acceptable salts thereof wherein:
A is a structure i, ii, iii, or iv; 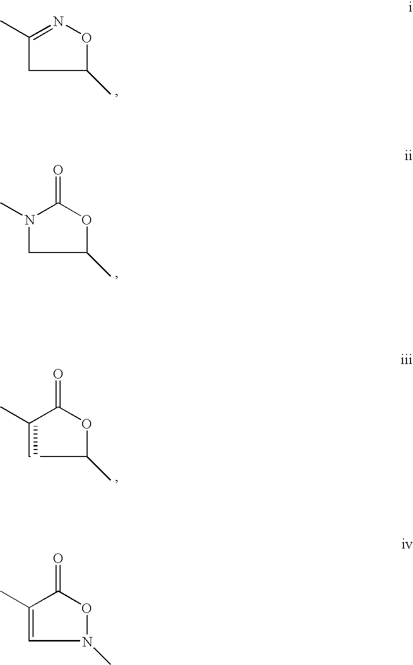 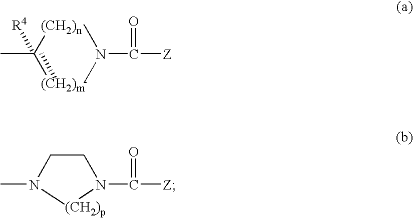
W is —N(H)C(═X)-R₁, Het, or —Y-HET, in which the Het or —Y-HET is optionally substituted with ═S or ═O;
X is O or S;
Y is NH, O, or S;
Z is
R₅—≡—(CH₂)ᵣ—E—
E is CH₂ or C═O;
R₁ is a) H,
b) NH₂,
c) NHC₁₋₄alkyl,
d) C₁₋₄alkyl,
e) C₂₋₄ alkenyl,
i) O—C₁₋₄alkyl,
j) S—C₁₋₄alkyl, or
k) (CH₂)ₛ C₃₋₆ cycloalkyl, in which each occurrence of alkyl or cycloalkyl in R₁ is optionally substituted by 1-3 halo;
Each R₂ is independently H, halogen, or C₁₋₄ alkyl;
R₄ is H, CH₃ or F;
R₅ is selected from H, aryl, and heteroaryl, each optionally substituted with 1-3 of R₆;
R₆ is halogen, (CH₂)ₘNHR₇, (CH₂)ₚR₇, CH₂—CHR₉—C(O)—R₈, OR₈, S(O)_{q}R₇, CN, C(═O)R₉, C(═NR₁₀)NHR₈, or C(═NR₁₀)R₈;
Each R₇ and R₈ is independently H, C₁₋₆ alkyl, aryl, or heteroaryl;
R₉ is OH, OR₈, C₁₋₆ alkyl, aryl, heteroaryl, or N(R₇)(R₈);
R₁₀ is OR₈ or N(R₇)(R₈);
m is 0, 1, 2, 3, 4;
n is 0, 1, 2, 3, 4 with the proviso that m plus n is 2, 3, 4, or 5;
p is 1, 2, 3;
q is 0, 1, 2;
r and s are independently 0, 1, 2, 3, 4, 5 or 6.

2. The compound of claim 1, wherein r is 1, 2, 3, or 4.

3. The compound of claim 1, wherein R₅ is phenyl optionally substituted with R₆.

4. The compound of claim 3, wherein R₆ is (CH₂)ₘNHR₇.

5. The compound of claim 4, wherein R₆ is —CH₂—NH₂.

6. The compound of claim 3, wherein R₆ is CH₂—CHR₉—C(═O)—R₈.

7. The compound of claim 6, wherein R₆ is —CH₂—CH(NH₂)—C(═O)—OH or —H₂—CH(NH₂)—C(═O)—O—CH₃.

8. The compound of claim 3, wherein R₆ is OR₈.

9. The compound of claim 8, wherein R₆ is −OH or —OCH₃.

10. The compound of claim 3, wherein R₆ is C(═O)R₉.

11. The compound of claim 10, wherein R₆ is —C(O)—CH₃.

12. The compound of claim 1, wherein B is b and p is 2.

13. The compound of claim 1, wherein at least one R₂ is H.

14. A compound selected from:
N-({(5S)-3-[4-(4-{6-[3-(aminomethyl)phenyl]hex-5-ynoyl} piperazin-1-yl)-3-fluorophenyl]-2-oxo-1,3-oxazolidin-5-yl}methyl)acetamide;
N-({(5S)-3-[3-fluoro-4-(4-hex-5-ynoylpiperazin-1-yl)phenyl]-2-oxo-1,3-oxazolidin-5-yl} methyl)acetamide;
N-({(5S)-3-[3-fluoro-4-(4-hept-6-ynoylpiperazin-1-yl)phenyl]-2-oxo-1,3-oxazolidin-5-yl} methyl)acetamide;
N-{[(5S)-3-(3-fluoro-4-{4-[5-(4-hydroxyphenyl)pent-4-ynoyl]piperazin-1-yl}phenyl)-2-oxo-1,3-oxazolidin-5-yl]methyl} acetamide;
N-{[(5S)-3-(3-fluoro-4-{4-[5-(3-hydroxyphenyl)pent-4-ynoyl]piperazin-1-yl}phenyl)-2-oxo-1,3-oxazolidin-5-yl]methyl} acetamide;
N-({(5S)-3-[4-(4-{5-[4-(aminomethyl)phenyl]pent-4-ynoyl} piperazin-1-yl)-3-fluorophenyl]-2-oxo-1,3-oxazolidin-5-yl}methyl)acetamide;
N-{[(5S)-3-(3-fluoro-4-{4-[6-(4-hydroxyphenyl)hex-5-ynoyl]piperazin-1-yl} phenyl)-2-oxo-1,3-oxazolidin-5-yl]methyl} acetamide;
N-{[(5S)-3-(3-fluoro-4-{4-[6-(3-hydroxyphenyl)hex-5-ynoyl]piperazin-1-yl}phenyl)-2-oxo-1,3-oxazolidin-5-yl]methyl} acetamide;
N-({((5S)-3-[4-(4-{6-[4-(aminomethyl)phenyl]hex-5-ynoyl} piperazin-1-yl)-3-fluorophenyl]-2-oxo-1,3-oxazolidin-5-yl} methyl)acetamide;
N-{[(5S)-3-(3-fluoro-4-{4-[7-(3-hydroxyphenyl)hept-6-ynoyl]piperazin-1-yl} phenyl)-2-oxo-1,3-oxazolidin-5-yl]methyl} acetamide;
N-{[(5S)-3-(3-fluoro-4-{4-[7-(4-hydroxyphenyl)hept-6-ynoyl]piperazin-1-yl} phenyl)-2-oxo-1,3-oxazolidin-5-yl]methyl} acetamide;
N-({(5S)-3-[4-(4-{7-[4-(aminomethyl)phenyl]hept-6-ynoyl} piperazin-1-yl)-3-fluorophenyl]-2-oxo-1,3-oxazolidin-5-yl} methyl)acetamide;
N-{[(5S)-3-(4-{4-[5-(2,4-dioxo-1,2,3,4-tetrahydropyrimidin-5-yl)pent-4-ynoyl]piperazin-1-yl}-3-fluorophenyl)-2-oxo-1,3-oxazolidin-5-yl]methyl} acetamide;
methyl 4-{5-[4-(4-{(5S)-5-[(acetylamino)methyl]-2-oxo-1,3-oxazolidin-3-yl}-2-fluorophenyl)piperazin-1-yl]-5-oxopent-1-ynyl}-L-phenylalaninate;
N-{[(5S)-3-(4-{4-[5-(4-aminophenyl)pent-4-ynoyl]piperazin-1-yl}-3-fluorophenyl)-2-oxo-1,3-oxazolidin-5-yl]methyl} acetamide;
N-[((5S)-3-{3-fluoro-4-[4-(6-{4-[(methylamino)methyl]phenyl} hex-5-ynoyl)piperazin-1-yl]phenyl}-2-oxo-1,3-oxazolidin-5-yl)methyl]acetamide;
N-({(5S)-3-[3-fluoro-4-(4-{6-[4-(1H-imidazol-1-ylmethyl)phenyl]hex-5-ynoyl} piperazin-1-yl)phenyl]-2-oxo-1,3-oxazolidin-5-yl} methyl)acetamide;
N-{[(5S)-3-(4-{4-[6-(4-acetylphenyl)hex-5-ynoyl]piperazin-1-yl}-3-fluorophenyl)-2-oxo-1,3-oxazolidin-5-yl]methyl} acetamide;
N-({(5S)-3-[4-(1-{6-[3-(aminomethyl)phenyl]hex-5-ynoyl}-3-methylazetidin-3-yl)phenyl]-2-oxo-1,3-oxazolidin-5-yl}methyl)propanamide;
N-[((5S)-3-{3-fluoro-4-[4-(6-{4-[(1E)-N-hydroxyethanimidoyl]phenyl} hex-5-ynoyl)piperazin-1-yl]phenyl}-2-oxo-1,3-oxazolidin-5-yl)methyl]acetamide;
N-{[(5S)-3-(4-{4-[6-(3-cyanophenyl)hex-5-ynoyl]piperazin-1-yl}-3-fluorophenyl)-2-oxo-1,3-oxazolidin-5-yl]methyl}acetamide;
4-{5-[4-(4-{(5S)-5-[(acetylamino)methyl]-2-oxo-1,3-oxazolidin-3-yl}-2-fluorophenyl)piperazin-1-yl]-5-oxopent-1-ynyl}-L-phenylalanine;
N-[((5S)-3-{4-[4-(6-{4-[(Z)-amino(hydroxyimino)methyl]phenyl}hex-5-ynoyl)piperazin-1-yl]-3-fluorophenyl}-2-oxo-1,3-oxazolidin-5-yl)methyl]acetamide hydrochloride;
N-({(5S)-3-[4-(4-{6-[3-(aminomethyl)phenyl]hex-5-ynoyl}piperazin-1-yl)-2,3,5-trifluorophenyl]-2-oxo-1,3-oxazolidin-5-yl}methyl)acetamide and N-({(5S)-3-[4-(4-{6-[4-(aminomethyl)phenyl]hex-5-ynoyl}piperazin-1-yl)-2,3,5-trifluorophenyl]-2-oxo-1,3-oxazolidin-5-yl} methyl)acetamide.

15. A method for the treatment of microbial infections in mammals comprising administration of an effective amount of compound of claim 1 to said mammal.

16. The method of claim 15 wherein said compound of claim 1 is administered to the mammal orally, parenterally, transdermally, or topically in a pharmaceutical composition.

17. The method of claim 16 wherein said compound is administered in an amount of from about 0.1 to about 100 mg/kg of body weight/day.

18. The method of claim 16 wherein said compound is administered in an amount of from about 1 to about 50 mg/kg of body weight/day.

19. A pharmaceutical composition comprising a compound of claim 1 and a pharmaceutically acceptable carrier.
